SEQUENCE LISTING (12 sequences)

SEQ ID NO: 1 - protein, 10 aa, Homo sapiens
GGTFNNNAIN

SEQ ID NO: 2 - protein, 17 aa, Homo sapiens
GIIPMFGTAKYSQNFQG

SEQ ID NO: 3 - protein, 14 aa, Homo sapiens
SRDLLLFPHHALSP

SEQ ID NO: 4 - protein, 11 aa, Homo sapiens
QGDSLRSYYAS

SEQ ID NO: 5 - protein, 7 aa, Homo sapiens
GKNNRPS

SEQ ID NO: 6 - protein, 11 aa, Homo sapiens
SSRDSSGNHWV

SEQ ID NO: 7 - protein, 123 aa, Homo sapiens
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 8 - protein, 109 aa, Homo sapiens
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 9 - protein, 453 aa, Homo sapiens
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo sapiens
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 12 - protein, 152 aa, Homo sapiens [sequence omitted]

(19) United States
(12) Patent Application Publication
(10) Pub. No.: US 2025/0306020 A1
(43) Pub. Date: Oct. 2, 2025

(54) BIOMARKERS

(71) Applicant: UCL BUSINESS LTD, London (GB)

(72) Inventors: Michael Ehrenstein, London (GB); Muhammad Shipa, London (GB)

(21) Appl. No.: 18/864,514

(22) PCT Filed: May 9, 2023
(86) PCT No.: PCT/GB2023/051212

§ 371 Date: Nov. 8, 2024

(30) Foreign Application Priority Data
May 10, 2022 (GB) 2206840.7

(51) Int. Cl. G01N 33/564; C07K 16/44; G01N 33/53

(52) U.S. Cl. CPC G01N 33/564; C07K 16/44; G01N 33/5308; G01N 2800/24

(57) ABSTRACT

The invention relates to the use of IgA2 antibodies, in particular IgA2 anti-dsDNA antibodies, as a biomarker. The antibodies may be use as a biomarker for autoimmune disease, such as SLE. The antibodies may also be used to identify a subject with an autoimmune disease who is likely to benefit from treatment with an agent which targets B-cell Activating Factor (BAFF).

### FIELD OF INVENTION

The invention relates to the use of IgA2 antibodies, for example IgA2 anti-dsDNA antibodies, as a biomarker, and in particular for use in identifying subjects who may benefit from specific treatment.

### BACKGROUND

Systemic lupus erythematosus (SLE) is one of a number of autoimmune diseases characterised by an array of clinical features and immune abnormalities. This heterogeneity is reflected by divergent responses to treatment, including to targeted therapies. Molecular and immunological phenotyping has stratified lupus patients into several major groups, which likely contributes to the heterogeneous clinical presentation, severity and outcome, and also may explain highly variable responses to targeted therapies. Reliance on generalised immunosuppression and systemic glucocorticoids remains, reflecting the lack of alternative effective therapies, and consequently the improvement in outcomes for lupus patients has slowed over recent years (van Vollenhoven, R. F. J. Intern. Med, 284, 228-239 (2018); Arnaud, L. & Tektonidou, M. G., Rheumatology 59, 29-38 (2020)). The production of autoantibodies against nuclear proteins, in particular double-stranded DNA (dsDNA) is a hallmark of SLE, and is associated with disease activity (Isenberg, D., Manson, J., Ehrenstein, M. & Rahman, A. Rheumatology, 46:7, 1052-1056, (2007); Floris, A., Piga, M., Cauli, A. & Mathieu, A., Autoimmunity Reviews 15, 656-663 (2016); Gordon, C., et al, Rheumatology 57, e1-e45 (2017); Carter, L. M., Isenberg, D. A. & Ehrenstein, M. R., Arthritis & Rheumatism 65, 2672-2679 (2013)). Evidence of pathogenicity of IgG anti-dsDNA antibody underscores their relevance and provides a persuasive rationale for B cell targeted therapies for SLE (Merrill, J. T., et al., Arthritis & Rheumatism 62, 222-233 (2010)).

The first widely used targeted therapy for SLE was rituximab, which targets the pan B cell marker CD20 and its administration results in a rapid depletion of B cells. It is frequently used for patients with SLE refractory to conventional therapy or requiring high dose steroids to control disease (Gordon, C., et al, Rheumatology 57, e1-e45 (2017): Fanouriakis, A., et al., Annals of the Rheumatic Diseases 78, 736-745 (2019)). However, there is a considerable variation in response to rituximab. The B cell activating factor (BAFF)-neutralizing monoclonal antibody belimumab was the first biologic licensed for the treatment of lupus following two large phase III clinical trials and has recently been shown to be effective for renal lupus (Furie, R., et al. New England Journal of Medicine 383, 1117-1128 (2020)).

Increased levels of the B cell cytokine BAFF and its association with worsening disease after rituximab, led to a randomised placebo controlled clinical trial (BEAT-LUPUS) comparing treatment with belimumab after rituximab, to rituximab alone. Results demonstrated that the combination significantly reduced serum IgG anti-DNA antibody levels and the risk of severe flares during the 52 weeks of treatment (Shipa, M., et al., Annals of Internal Medicine, Oct. 26 (2021)). A variation in response highlights further the need for biomarkers to target those most likely to respond.

Thus, there remains a need to stratify patients, to aid treatment selection and reduce costs and burden of unnecessary or ineffective treatments, and therefore to improve clinical outcome, given the immunopathological and clinical complexity of SLE combined with variability of response to therapy.

### SUMMARY

In an aspect, the invention provides IgA2 antibodies, in particular IgA2 anti-dsDNA antibodies, for use as a biomarker.

In another aspect, the invention provides IgA2 antibodies, in particular IgA2 anti-dsDNA antibodies, for use as a biomarker in therapy.

In another aspect, the invention provides IgA2 antibodies for use as a biomarker in the treatment or diagnosis of an autoimmune disease in a subject who is identified as likely to benefit from treatment, using a method described herein. In a particular aspect, the IgA2 antibodies are IgA2 anti-dsDNA antibodies.

In another aspect, the invention provides an agent which targets B-cell Activating Factor (BAFF) for use in the treatment of an autoimmune disease in a subject who is identified as likely to benefit from treatment. The subject may be identified by a method described herein.

In another aspect, the invention provides a method of identifying a subject with an autoimmune disease who is likely to benefit from treatment with an agent which targets B-cell Activating Factor (BAFF), comprising:
i. providing a biological sample obtained from the subject;
ii. determining the level of IgA2 antibodies, such as IgA2 anti-dsDNA antibodies, in the sample;
iii. using the results from step (ii) to determine if the subject is likely to benefit from treatment with an agent which targets BAFF.

In another aspect, there is provided a method of treating a subject with an autoimmune disease, such as SLE, comprising:
i. providing a biological sample obtained from the subject;
ii. determining the level of IgA2 antibodies, such as IgA2 anti-dsDNA antibodies, in the sample;
iii. using the results from (ii) to determine if the subject is likely to benefit from treatment with an agent which targets BAFF; and
iv. administering an agent which targets BAFF to the subject if the subject is determined to have an autoimmune disease.

In a further aspect, there is provided a method of treating an autoimmune disease, such as SLE, in a subject having an increased level of IgA2 antibodies, such as IgA2 anti-dsDNA antibodies, comprising administration to the subject of a therapeutically effective amount of an agent which targets BAFF. The subject may also be administered simultaneously, sequentially, or separately, an agent which targets CD20.

In another aspect, there is provided a method of treating an autoimmune disease, such as SLE, in a subject, wherein the method comprises identifying a subject having an increased level of IgA2 antibodies, such as IgA2 anti-dsDNA antibodies, and administering to the identified subject a therapeutically effective amount of an agent which targets BAFF. The subject may also be administered simultaneously, sequentially, or separately, an agent which targets CD20.

In another aspect, there is provided a method of treating an autoimmune disease, such as SLE, in a subject classified as a responder, wherein a responder is characterised by an increased level of IgA2 antibodies, such as IgA2 anti-dsDNA antibodies, compared to a reference sample, comprising administering to the identified subject a therapeutically effective amount of an agent which targets BAFF. The subject may also be administered simultaneously, sequentially, or separately, an agent which targets CD20.

In another aspect, there is provided a kit comprising one or more probes capable of binding to IgA2 antibodies, such as IgA2 anti-dsDNA antibodies, optionally wherein the kit further comprises a set of instructions. The kit may further comprise components for performing an ELISA.

### DETAILED DESCRIPTION

In any aspect, a subject may be determined as likely to benefit from treatment with an agent which targets BAFF, when the level of IgA2 antibodies, such as IgA2 anti-dsDNA antibodies, is increased compared to the level of IgA2 antibodies, such as IgA2 anti-dsDNA antibodies, in a reference sample. The level of IgA2 antibodies, such as IgA2 anti-dsDNA antibodies, may be increased by about two-fold or more, such as about three-fold or more, about four-fold or more, about five-fold or more, compared to the level of IgA2 antibodies, such as IgA2 anti-dsDNA antibodies, in a reference sample.

Alternatively, a subject may be determined as likely to benefit from treatment with an agent which targets BAFF, when the level of IgA2 antibodies, such as IgA2 anti-dsDNA antibodies, is increased by about 10%, 25%, 50%, 75%, 100%, 200%, 500% or 1000% compared to the level of IgA2 antibodies, such as IgA2 anti-dsDNA antibodies, in a reference sample.

Alternatively, a subject may be determined as likely to benefit from treatment with an agent which targets BAFF, when IgA2 antibodies, such as IgA2 anti-dsDNA antibodies, are found in a sample from the subject and the reference sample does not contain IgA2 antibodies, such as IgA2 anti-dsDNA antibodies.

In any aspect, a subject may be determined as likely to benefit from treatment with an agent which targets BAFF, when an Optical Density (OD) of about 0.1 or more, 0.2 or more, 0.3 or more, 0.4 or more, 0.5 or more, 0.6 or more, 0.7 or more, or 0.8 or more is calculated for the level of IgA2 antibodies, such as IgA2 anti-dsDNA antibodies in a sample, for example by ELISA.

A subject may be determined as likely to benefit from treatment with an agent which targets BAFF, when an Optical Density (OD) of about 0.10 or more, 0.11 or more, 0.12 or more, 0.13 or more, 0.14 or more, 0.15 or more, 0.16 or more, 0.17 or more, 0.18 or more, 0.19 or more, 0.20 or more is calculated for the level of IgA2 antibodies, such as IgA2 anti-dsDNA antibodies in a sample, for example when measured by ELISA.

A subject may be determined as likely to benefit from treatment with an agent which targets BAFF, when an Optical Density (OD) of about 0.3 or more, 0.5 or more, 0.8 or more, 0.9 or more, 1.0 or more, 1.2 or more, 1.5 or more is calculated for the level of IgA2 antibodies, such as IgA2 anti-dsDNA antibodies in a sample, for example when measured by ELISA.

A subject may be determined as likely to benefit from treatment with an agent which targets BAFF, when an Optical Density (OD) of about 0.30 or more, 0.32 or more, 0.33 or more, 0.34 or more, 0.50 or more, 0.78 or more, 0.92 or more, 1.00 or more 1.02 or more, 1.15 or more, 1.45 or more is calculated for the level of IgA2 antibodies, such as IgA2 anti-dsDNA antibodies in a sample, for example when measured by ELISA.

A subject may be determined as likely to benefit from treatment with an agent which targets BAFF, when an Optical Density (OD) of between about 0.2 and about 0.8, between about 0.3 and about 0.8, between about 0.2 and about 0.5, is calculated for the level of IgA2 antibodies, such as IgA2 anti-dsDNA antibodies, in a sample, for example when measured by ELISA.

In any aspect, the reference sample may refer to an equivalent sample taken from a healthy subject, or the average value of a given parameter when an equivalent sample is taken from more than one healthy subject.

In any aspect, a method of the invention may also identify a subject who is likely to benefit from treatment with an agent which targets B-cell Activating Factor (BAFF) and an agent which targets CD20.

In any aspect, a subject who is identified as likely to benefit from treatment with an agent which targets B-cell Activating Factor (BAFF), may also be treated with an agent which targets CD20.

In an embodiment, the subject may be treated with the agent which targets CD20 before being treated with the agent which targets BAFF.

In an embodiment, the agent which targets CD20 is administered less than four weeks before the agent which targets BAFF, between four to eight weeks before the agent which targets BAFF, or 8-12 weeks before the agent which targets BAFF.

In an embodiment, the subject may be treated with the agent which targets CD20 at the same time as being treated with the agent which targets BAFF. If administered at the same time, the agent which targets BAFF and the agent which targets CD20 may be in the same formulation, or alternatively the agents may be in different formulations which are administered simultaneously or sequentially.

In an embodiment, the subject may be treated with the agent which targets CD20 after being treated with the agent which targets BAFF.

In an embodiment, the agent which targets CD20 is administered at least two weeks after the agent which targets BAFF.

In an embodiment, the agent which targets CD20 is administered at least twice between weeks 2 and 20 after the agent which targets BAFF was administered. For example, the agent which targets CD20 is administered at least at weeks 2 and 20, weeks 4 and 18, weeks 6 and 16, weeks 8 and 14 or weeks 10 and 12 after the agent which targets BAFF.

In any aspect, the step of determining the level of IgA2 antibodies, such as IgA2 anti-dsDNA antibodies, may be performed before the agent which targets BAFF has been administered. When the subject is to be treated with an agent which targets CD20, the step of determining the level of IgA2 antibodies, such as IgA2 anti-dsDNA antibodies, may also be performed before the agent which targets CD20 has been administered.

In any aspect, the autoimmune disease may be systemic lupus erythematosus (SLE), lupus nephritis or cutaneous Lupus.

In any aspect the autoimmune disease may be Sjogren's syndrome, inflammatory bowel disease, connective tissue disease associated interstitial lung disease, ankylosing spondylitis autoimmune hepatitis, IgA-nephropathy, rheumatoid arthritis, coeliac disease, vasculitis, Systemic Sclerosis, idiopathic membranous nephropathy, antiphospholipid syndrome, IgG4 related disease, graft versus host disease (GvHD), idiopathic thrombocytopenia purpura or multiple sclerosis. In these autoimmune diseases, the IgA2 antibody is specific for autoantigen(s) associated with the autoimmune disease. IgA autoantibodies have been described for various autoimmune diseases such as inflammatory bowel disease, IgA nephropathy, IgA vasculitis, celiac disease, ankylosing spondylitis, alcoholic liver cirrhosis and rheumatoid arthritis. For instance, Anti-Neutrophil Cytoplasm Antibodies (ANCA)-associated vasculitis patients usually have autoantibodies against PR3 (PR3-ANCA) or MPO (MPO-ANCA). Thus in the context of ANCA-associated vasculitis, levels of IgA2 anti-PR3 or IgA2 anti-MPO antibodies may be indicative if the subject is likely to benefit from treatment with an agent which targets BAFF

In any aspect, a subject may be taken as benefitting from treatment when one or more symptom(s) of the autoimmune disease, such as SLE is alleviated by treatment. A subject may be taken as benefitting from treatment when a major clinical response (MCR) is achieved 52 weeks after beginning treatment. MCR is defined as a reduction in BILAG-2004 (BILAG stands for British Isles Lupus Assessment Group) index A or B scores to BILAG-2004 C (or D) in all domains, a reduction in steroid dose to ≤7.5 mg daily and a modified SLEDAI-2K score≤2 (without including the anti-dsDNA antibody component).

As used herein, an “agent which targets B-cell Activating Factor (BAFF)” refers to an agent which may bind to, reduce or inhibit the activity of, and/or reduce or inhibit the expression of BAFF. The skilled person will understand that various techniques are available in the art to determine whether the agent has had such an effect on BAFF.

As used herein, an “agent which targets CD20” refers to an agent which may bind to, reduce or inhibit the activity of, and/or reduce or inhibit the expression of CD20, or to reduce the B-cell number. The skilled person will understand that various techniques are available in the art to determine whether the agent has had such an effect on CD20 or B-cell numbers. In an alternative embodiment of any aspect, an agent which targets CD19, or any other B cell specific marker, may be used in place of, or in addition to, an agent which targets CD20.

In any aspect, an “agent” may refer to an antigen binding protein, such as a CAR T-cell or antibody, nucleic acid, and/or small molecule. An antigen binding protein may be an antibody. An agent which is a nucleic acid may be a DNA or RNA molecule. A nucleic acid may be an siRNA or an shRNA. A small molecule may be a small molecule inhibitor.

The agent which targets BAFF may be an antigen binding protein. The antigen binding protein may be an antibody or CAR T-cell, for example.

The antigen binding protein which targets BAFF may comprise:
(a) a heavy chain variable domain comprising:
   i. a CDR1 comprising or consisting of SEQ ID NO: 1;
   ii. a CDR2 comprising or consisting of SEQ ID NO: 2; and
   iii. a CDR3 comprising or consisting of SEQ ID NO: 3, and/or
(b) a light chain variable domain comprising:
   i. a CDR1 comprising or consisting of SEQ ID NO: 4;
   ii. a CDR2 comprising or consisting of SEQ ID NO:5; and
   iii. a CDR3 comprising or consisting of SEQ ID NO:6,
      or a sequence with at least about 90% or more, such as 90%, 95%, 98%, 99% identity to one or more of SEQ ID NOs: 1-6.

The antigen binding protein which targets BAFF may comprise:
(a) a variable heavy chain sequence comprising or consisting of SEQ ID NO: 7; and
(b) a variable heavy chain sequence comprising or consisting of SEQ ID NO: 8, or a sequence with at least about 90% or more, such as 90%, 95%, 98%, 99% identity to one or more of SEQ ID NOs: 7-8.

The antigen binding protein which targets BAFF may comprise or consist of:
(a) a heavy chain sequence comprising or consisting of SEQ ID NO: 9; and
(b) a light chain sequence comprising or consisting of SEQ ID NO: 10, or a sequence with at least about 90% or more, such as 90%, 95%, 98%, 99% identity to one or more of SEQ ID Nos: 9-10.

The antigen binding protein which targets BAFF may be the antibody belimumab.

Preferably, the agent which targets CD20 is an antigen binding protein, preferably an antibody. The antibody may be an anti-CD20 antibody, such as rituximab, ocrelizumab, ofatumumab, ublituximab, veltuzumab, obinutuzumab, ocaratuzumab, PRO131921, tositumomab, or ibritumomab. Preferably, the anti-CD20 antibody is rituximab.

In a preferred embodiment of any aspect, the subject is a human, and the autoimmune disease is SLE, and the agent which targets BAFF is belimumab.

In any aspect, the biological sample may be a blood or serum sample, or a urine sample. Preferably the sample is a serum sample.

Any suitable assay may be used to perform the step of determining the level of IgA2 antibodies, such as IgA2 anti-dsDNA antibodies.

When determining the level of IgA2 antibodies, such as IgA2 anti-dsDNA antibodies, in the biological sample, an ELISA may be used.

As disclosed herein, “BAFF” may also be referred to as B lymphocyte stimulator protein (BLyS).

The one or more probe may be a binding agent that is capable of specific/selective binding to IgA2 anti-dsDNA antibodies. The one or more probe may comprise or consist of a polypeptide and/or nucleic acid, such as DNA. The one or more probe may comprise or consist of an antibody, an antibody variant or memetic, or a binding-fragment thereof. The one or more probe may comprise or consist of an aptamer. The one or more probe may be a polyclonal or monoclonal antibody, or fragment thereof. The antibody, or fragment thereof, may be of any mammalian species, such as human, simian, porcine, camelid or rabbit.

The probe or probes may be immobilised on a substrate. One or more, or all of the probes may be anchored to a surface, such as the surface of a solid substrate. The solid substrate may be a plate, such as a microwell plate. The solid substrate may be a particle, such as a nano- or micro-particle. In one embodiment the solid substrate is a bead.

The one or more probe may comprise a tag identification and/or capture. The tag may comprise a fluorescent molecule, or an enzyme. The probe may be radiolabelled.

Where ELISA, or similar assay, is used, the probe may be a primary antibody for binding to the target, and a secondary tagged-antibody probe may be provided for binding to the primary antibody or the biomarker for detection.

The term “antibody” includes substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments, antibody mimetics, and derivatives of the same. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It has been shown that fragments of a whole antibody can perform the function of binding antigens. Examples of binding fragments of the invention are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers and; (ix) “diabodies”, multivalent or multispecific fragments constructed by gene fusion.

In any aspect, the subject is a mammal. Preferably, the subject is human.

As used herein “screening” refers to the first screening visit before any treatment is administered. The term “baseline” refers to the parameter measured at the time of screening.

The skilled person will understand that optional features of one embodiment or aspect of the invention may be applicable, where appropriate, to other embodiments or aspects of the invention.

In an embodiment, the inventors have identified serum IgA2 anti-dsDNA antibodies as a strong positive predictor of response to treatment with the anti BAFF antibody belimumab.

In another embodiment the inventors have identified serum IgA2 anti-dsDNA antibodies as a strong positive predictor of response to treatment with the anti BAFF antibody belimumab in combination with treatment with the anti-CD20 antibody rituximab in SLE. IgA2 outperformed other anti-DNA antibody isotypes, including IgG anti-dsDNA antibody, in predicting response to treatment. The data presented herein shows that IgA2 antibodies, such as IgA2 anti-dsDNA antibodies, can be used as a biomarker to stratify patients with autoimmune diseases such as SLE, to provide administration of appropriate treatments and to improve on the modest outcomes that are currently achieved.

Embodiments of the invention will now be described in more detail, by way of example only, with reference to the accompanying drawings.

### DESCRIPTION OF THE DRAWINGS

FIG. 1—shows baseline predictors of major clinical response to belimumab following rituximab, and placebo following rituximab, at 52 weeks.

Belimumab: (a) Sparse Partial Least Squares Discriminant Analysis (sPLS-DA)—Factor-loading weights in component 1 are shown for the top 5 (chosen by model optimisation) ranked parameters to predict belimumab response at 52 weeks. (b) Forest plot-odds ratios (OR) with 95% confidence interval (95% CI), by univariate logistic regression^{†}, of the variables chosen by sPLS-DA with p values. (c) Multiple logistic regression^{†} to construct the final model to predict belimumab response at 52 weeks, where variables were selected by random forest classification algorithm; with Area under the Receiver operator characteristic (ROC) curve (AUROC) of this final model to predict belimumab response. Placebo: (d) sPLS-DA—Factor-loading weights in component 1 are shown for the top 5 (chosen by model optimisation) ranked parameters to predict placebo response at 52 weeks. (e) Forest plot—OR with 95% CI of the predictors chosen by sPLS-DA with p values. (f) Multiple logistic regression^{†} to construct the final model to predict placebo response at 52 weeks, where variables were selected by random forest classification algorithm; with AUROC of this final model to predict placebo response.^{†} Unit changes for the continuous variables used in the logistic regression are shown in table 1. IFN=Interferon. Ig=Immunoglobulin. IL=interleukin, TNF=Tumour necrosis factor.

FIG. 2—demonstrates that increased baseline serum IgA2 anti-dsDNA antibody levels both predict major clinical response at 52 weeks and fall during treatment with belimumab after rituximab. (a) Area under the Receiver operator characteristic (ROC) curve (AUROC) of serum IgA2 anti-dsDNA antibodies at baseline to predict treatment response to belimumab and placebo at 52 weeks. Longitudinal change of serum IgA2 anti-dsDNA antibodies (in OD=optical density) stratified by—(b) treatment i.e., belimumab versus placebo (after rituximab). (c) treatment response in belimumab treated group, and (d) treatment response in placebo treated group. A longitudinal linear mixed-effect model was fitted with random effect of patient ID and fixed effect of treatment group intercepting with trial times and adjusted for screening IgA2 anti-dsDNA antibody values, age, gender, concomitant mycophenolate (yes or no), and prednisolone dose at respective time points to calculate expected difference at 24 and 52 weeks in serum IgA2 anti-dsDNA antibodies. Estimated mean with 95% confidence intervals and number of patients at each time points (n) are shown; p values at weeks 24 and 52 are provided. Black Dotted line indicates upper limit of normal (3 standard deviation above the mean of healthy control samples).* All the samples were available from screening to 24 weeks, except one sample was unavailable at 52 weeks.

FIG. 3—demonstrates that elevated IgA2 producing plasmablasts in peripheral blood at baseline predict major clinical response to belimumab after rituximab, and decrease with treatment. Representative flow cytometry plots of IgA1 and IgA2 secreting plasmablasts (gated/defined as CD19⁺CD27^{hi}CD38^{hi}) at screening and 52 weeks stratified by (a) belimumab (after rituximab) responders and non-responders and (b) placebo (after rituximab) responders and non-responders. Cumulative data of percentage and absolute numbers of IgA2 secreting plasmablasts at screening and 52 weeks are stratified by—(c) responders and non-responders in belimumab arm, (d) responders and non-responders in placebo arm, and (e) their exposed treatment either to belimumab or placebo. A linear mixed-effect model was fitted with random effect of patient ID and fixed effect of treatment group intercepting with trial times between screening and week 52 and adjusted for age, gender, concomitant mycophenolate (yes or no), and prednisolone dose at the two time points to calculate expected difference at 52 weeks. Estimated mean with 95% confidence intervals and number of patients (n) at the two time-points are shown; p value at weeks 52 is provided. Comparison between responders and non-responders (by Mann-Whitney's U test) to belimumab by—(f) percentage of IgA2 secreting plasmablasts and (g) absolute number of IgA2 secreting plasmablasts at screening, and to placebo by—(h) percentage of IgA2 secreting plasmablasts and (i) absolute number of IgA2 secreting plasmablasts at screening.

FIG. 4—shows that increased baseline T-follicular helper cells predict clinical response to belimumab after rituximab and decrease with treatment. (a) Representative flow cytometry plots of Tfh (defined as CXCR5⁺ICOS⁺PD-1⁺) at screening and 52 weeks stratified by treatment with belimumab and placebo. Cumulative percentage and absolute number of Tfh at screening and 52 weeks are shown stratified by—(b) treatment with belimumab or placebo, (c) responders and non-responders in the belimumab arm, and (d) responders and non-responders in the placebo arm. A longitudinal linear mixed-effect model was fitted with random effect of patient ID and fixed effect of treatment group intercepting with trial times and adjusted for screening Tfh values, age, gender, concomitant mycophenolate (yes or no), and prednisolone dose at respective time points to calculate expected difference at 24 and 52 weeks in Tfh. Estimated mean with 95% confidence intervals and number of patients at each time points (n) are shown; p values are provided. Comparison between responders and non-responders (by Mann-Whitney's U test) to belimumab by—(e) percentage of Tfh and (f) absolute number of Tfh at screening. Comparison between responders and non-responders to placebo by—(g) percentage of Tfh and (h) absolute number of Tfh at screening.

FIG. 5—demonstrates that Serum IgA2 anti-dsDNA antibody levels emerge as the strongest predictor of active renal disease at screening in contrast to serum IgA1 anti-dsDNA antibody and interferon as the most important predictors of active mucocutaneous disease. (a) Sparse Partial Least Squares Discriminant Analysis (sPLS-DA)—Factor-loading weights in component 1 are shown for the top-10 ranked parameters that predict active renal disease at baseline (screening). (b) Forest plot—Odds ratio (OR) with 95% confidence interval (95% CI) of multiple logistic regression^{†} to construct the final model to predict active renal disease at screening, where variables were selected by the random forest classification algorithm. (c) sPLS-DA—Factor-loading weights in component 1 are shown for the top-10 ranked parameters that predict active mucocutaneous disease at baseline (screening). (d) Forest plot—OR with 95% CI of multiple logistic regression^{†} to construct the final model to predict active mucocutaneous disease at screening, where variables were selected by the random forest classification algorithm.^{†} Unit changes for the continuous variables used in the logistic regression are shown in table 1. BAFF=B-cell activating factor. IFN=Interferon, IFN-I score=Type 1 interferon score, IFN-I A score=Type 1 A interferon score. IFN-I B score=Type 1 B interferon score, Ig=Immunoglobulin, IL=interleukin.

FIG. 6—shows baseline predictors of major clinical response to belimumab following rituximab, and placebo following rituximab, at 52 weeks. Belimumab: (a) Forest plot—odds ratios (OR) with 95% confidence interval (95% CI), by univariate logistic regression^{†} [3 of the 5 variables, chosen by Partial Least Squares Discriminant Analysis (sPLS-DA), with missing values]. (b) Regularised Random Forest (RRF)—Top 10 variables by mean decrease in Gini (ranked) represents the importance of each variable to predict the belimumab response. (c) Multiple logistic regression^{†} (complete case) to construct the final model to predict belimumab response at 52 weeks where variables were selected by random forest classification algorithm. Placebo: (d) Forest plot—OR with 95% confidence interval (95% CI), by univariate logistic regression^{†} [3 of the 5 variables, chosen by sPLS-DA, with missing values]. (c) Regularised Random Forest (RRF)—Top 10 variables by mean decrease in Gini (ranked) represents the importance of each variable to predict the placebo response. (f) Multiple logistic regression^{†} (complete case) to construct the final model to predict placebo response at 52 weeks where variables were selected by random forest classification algorithm.^{†} Unit changes for the continuous variables used in the logistic regression are shown in table 1. BAFF=B-cell activating factor, ESR=Erythrocyte sedimentation rate, IFN=Interferon, IFN-I B score=Type I B interferon score, Ig=Immunoglobulin, IL=interleukin, OBB=out of bag error, TNF=Tumour necrosis factor.

FIG. 7—shows that the percentage of patients with (a) positive^{†} IgA2 anti-dsDNA antibodies is significantly reduced at 52 weeks after belimumab (and rituximab) therapy, but not the percentage of patients with (b) positive^{††} IgG anti-dsDNA antibody. Comparison at 52 weeks was done by fisher-exact test and the p value is shown. ^{†}Positivity was defined as 3 standard deviation above the mean values from healthy controls (0.06 optical density). ^{††}Positivity was defined as per the manufacturer provided value (20 IU/ml).

FIG. 8—shows the change in serum total IgA2 (immunoglobulin A2) between screening and 52 weeks. Total IgA2 levels from screening to 52 weeks stratified by—(a) treatment either to belimumab or placebo (after rituximab), (b) major clinical responders and non-responders in the belimumab arm, and (c) responders and non-responders in the placebo arm. A longitudinal linear mixed-effect model was fitted with random effect of patient ID and fixed effect of treatment group intercepting with trial times and adjusted for screening value, age, gender, concomitant mycophenolate (yes or no), and prednisolone dose at the indicated time points to calculate expected difference at 24 and 52 weeks. Estimated mean with 95% confidence intervals and number of patients at each time points (n) are shown, and the p values at weeks 24 and 52 are provided. The black dotted line indicates the lower limit of the normal (as per the manufacturer).* All the samples were available from screening to 24 weeks, except one sample was unavailable at 52 weeks.

FIG. 9—demonstrates no change in serum IgA1 anti-dsDNA antibody between screening and 52 weeks. Serum IgA1 anti-dsDNA antibody levels (in OD=optical density) from screening to 52 weeks stratified by—(a) treatment either to belimumab or placebo (after rituximab), (b) major clinical responders and non-responders in the belimumab arm, and (c) responders and non-responders in the placebo arm. Longitudinal linear mixed-effect model was fitted with random effect of patient ID and fixed effect of treatment group intercepting with trial times and adjusted for screening value, age, gender, concomitant mycophenolate (yes or no), and prednisolone dose at indicated time points to calculate expected difference at 24 and 52 weeks. Estimated mean with 95% confidence intervals and number of patients at each time points (n) are shown, and the p values at weeks 24 and 52 are provided. The black dotted line indicates the upper limit of the normal (3 standard deviation above the mean of healthy control samples).* All the samples were available from screening to 24 weeks, except one sample was unavailable at 52 weeks.

FIG. 10—shows a change in serum IgG anti-dsDNA antibody between screening and 52 weeks. Serum IgG from screening to 52 weeks stratified by—(a) treatment either to belimumab or placebo (after rituximab), (b) major clinical responders and non-responders in the belimumab arm, and (c) responders and non-responders in placebo arm. Longitudinal linear mixed-effect model was fitted with random effect of patient ID and fixed effect of treatment group intercepting with trial times and adjusted for screening value, age, gender, concomitant mycophenolate (yes or no), and prednisolone dose at respective time points to calculate expected difference at 24 and 52 weeks. Estimated mean with 95% confidence intervals and number of patients at each time points (n) are shown, and the p values at weeks 24 and 52 are provided. The black dotted line indicates the upper limit of the normal (as per the manufacturer provided value).

FIG. 11—shows the flow cytometry gating strategy for detecting plasmablasts in the peripheral blood (defined as CD19⁺CD27^{hi}CD38^{hi}) and IgA1 and IgA2 secreting plasmablasts (by intracellular IgA1 and IgA2 staining). Lymphocytes were gated on FSC-A vs SSC-A and single cells identified. Dead cells were excluded using the blue Live/Dead stain. B cells were selected based on CD19 expression Plasmablast population were defined as CD19⁺CD27^{hi}CD38^{hi}. Anti-IgA1 and IgA2 intracellular staining was used to identify IgA1 and IgA2 secreting plasmablasts.

FIG. 12—shows the change in plasmablast frequency and absolute number in peripheral blood during the trial. (a) Representative flow cytometry plots of total plasmablasts (gated/defined as CD19⁺CD27^{hi}CD38^{hi}) at screening and 52 weeks stratified by belimumab (after rituximab) and placebo (after rituximab). Cumulative data of percentage and absolute numbers of plasmablasts at screening and 52 weeks (b) stratified by treatment and (c) belimumab (after rituximab) major clinical responders and non-responders. (d) Comparison between responders and non-responders (by Mann-Whitney's U test) to belimumab by—percentage of plasmablasts and absolute number of plasmablasts at screening. (e) Cumulative data of percentage and absolute numbers of plasmablasts at screening and 52 weeks are stratified by placebo (after rituximab) responders and non-responders. (f) Comparison between responders and non-responders (by Mann-Whitney's U test) to placebo by—percentage of plasmablasts and absolute number of plasmablasts at screening. A linear mixed-effect model was fitted with random effect of patient ID and fixed effect of treatment group intercepting with trial times between screening and Week 52 and adjusted for age, gender, concomitant mycophenolate (yes or no), and prednisolone dose at the two time points to calculate expected difference at 52 weeks. Estimated mean with 95% confidence intervals and number of patients (n) at the two time-points are shown; p value at 52 is shown.

FIG. 13—shows the gating strategy for T-follicular helper cells (defined as CD4⁺CXCR5⁺ICOS⁺PD-1⁺). Lymphocytes were gated on FSC-A vs SSC-A and single cells identified. Dead cells were excluded using the blue Live/Dead stain. T-follicular helper cells (Tfh) were defined as CD4⁺CXCR5⁺ICOS⁺PD-1⁺.

FIG. 14—shows correlation plots (Spearman's correlations) between (a) serum IgA2 anti-dsDNA antibody and % IgA2+ plasmablasts (defined as CD19⁺CD27^{hi}CD38^{hi}), (b) Tfh/T-follicular helper cell (defined as CD4⁺CXCR5⁺ICOS⁺PD-1⁺) and % IgA2+plasmablasts, (c) between serum IgA2 anti-dsDNA antibody and Tfh. and (d) between scrum IgA2 anti-dsDNA antibody levels and serum total IgA2.

FIG. 15—demonstrates the response from baseline through to 52 weeks to belimumab (after rituximab) and placebo (after rituximab) therapy stratified by organ involvement. (a) Odds ratio (OR) with 95% confidence interval (95% CI) of major clinical response (MCR), renal response, renal response with no new renal BILAG-2004 A/B flare, mucocutaneous response^{††}, and musculoskeletal response^{†††} at 52 weeks to belimumab (blue favours belimumab response) and placebo (red favours placebo response), by unadjusted univariable logistic regression. OR of cither BILAG-2004 A/B in placebo versus belimumab with 95% CI in (b) renal domain, (c) mucocutaneous domain, and (d) musculoskeletal domain from randomisation (rand.) through to 52 weeks. An unadjusted generalised linear mixed-effect model was applied to estimated OR with fixed effect of treatment group or treatment response intercepting with trial times, and random effect of within-patient.

FIG. 16—outlines parameters to predict active renal disease at screening. (a) Forest plot—odds ratios (OR) with 95% confidence by univariate logistic regression^{†} of the top 10-parameters selected by Sparse Partial Least Squares Discriminant Analysis (sPLS-DA). (b) univariate logistic regression of the parameters with missing value at screening among these 10-parameters. (c) Regularised Random Forest (RRF)—Top 10 variables by mean decrease in Gini (ranked) represents the importance of each variable to predict active renal disease.

FIG. 17—shows that serum IgA2 and IgM anti-dsDNA antibodies are biomarkers of active renal disease, with changes shown during the BEAT-lupus trial stratified by treatment (belimumab vs placebo both after rituximab), and renal response. (a) Longitudinal change in serum IgA2 anti-dsDNA antibody levels in OD (optical density) for individual patients through to 52 weeks-stratified by treatment and renal response for those patients who had active renal disease at screening from the BEAT-LUPUS trial. (b) Longitudinal change in serum IgM anti-dsDNA antibody levels (for individual patients) through to 52 weeks—stratified by treatment and renal response for those patients who had active renal disease at screening. The black solid lines (a-b) represent the estimated mean (utilising locally estimated scatterplot smoothing or LOESS), and the grey area represents the standard error. Dotted line in IgA2 anti-dsDNA antibody indicates upper limit of normal (For IgA2 anti-dsDNA antibody=3 standard deviation above mean of healthy donor samples, for IgM anti-dsDNA antibody=upper limit of normal provided by manufacturer).

FIG. 18—shows that serum IgM anti-dsDNA antibody is increased in belimumab (after rituximab) arm and accompanied by major clinical response. Longitudinal change of serum IgM anti-dsDNA antibodies stratified by—(a) treatment i.e., belimumab versus placebo (after rituximab), (b) treatment response in belimumab treated group, and (c) treatment response in placebo treated group. Longitudinal linear mixed-effect model was fitted with random effect of patient ID and fixed effect of treatment group intercepting with trial times and adjusted for screening IgM anti-dsDNA antibody values, age, gender, concomitant mycophenolate (yes or no), and prednisolone dose at respective time points to calculate expected difference at 24 and 52 weeks in IgM anti-dsDNA. Estimated mean with 95% confidence intervals and number of patients at each time points (n) are shown; p values at weeks 24 and 52 are provided. The black dotted line indicates the upper limit of the normal (as per the manufacturer provided value).

FIG. 19—shows that serum IgA2 and IgM anti-dsDNA antibodies are elevated in active renal disease. (a) BEAT-LUPUS samples and (b) validation cohort. P values are shown above by non-parametric Mann-Whitney U test. Dotted line indicates upper limit of normal (For IgA2 anti-dsDNA antibody=3 standard deviation above mean of healthy donor samples, for IgM anti-dsDNA antibody=upper limit of normal provided by manufacturer).

FIG. 20—outlines parameters to predict active mucocutaneous disease at screening. (a) Forest plot-odds ratios (OR) with 95% confidence by univariate logistic regression^{†} of the top 10-parameters selected by Sparse Partial Least Squares Discriminant Analysis (sPLS-DA). (b) univariate logistic regression^{†} of the parameters with missing value at screening among these 10-parameters. (c) Regularised Random Forest (RRF)—Top 10 variables by mean decrease in Gini (ranked) represents the importance of each variable to predict active renal disease. (d) multivariate logistic regression^{†} of the 3-parameters chosen by random forest classification algorithm (complete case).

FIG. 21—shows that serum IgA1 anti-dsDNA antibodies are elevated in active mucocutaneous disease. (a) BEAT-LUPUS samples and (b) validation cohort. P values are shown above by non-parametric Mann-Whitney U test. Dotted line indicates upper limit of normal (3 standard deviation above mean of healthy donor samples).

FIG. 22—demonstrates no change in (a) Interferon (IFN) type I score (total), (b) type I A score, and (c) serum IFN-α (log-transformed) from screening to 52 weeks stratified by the treatment either to belimumab or placebo (after rituximab). A linear mixed-effect model was fitted with random effect of patient ID and fixed effect of treatment group intercepting with trial times and adjusted for age, gender, concomitant mycophenolate (yes or no), and prednisolone dose at respective time points to calculate expected difference at 24 and 52 weeks. Estimated mean with 95% confidence intervals and number of patients at each time points (n) are shown, and the p values at 52 are provided.

FIG. 23—shows predictors of active musculoskeletal disease at screening. (a) Sparse Partial Least Squares Discriminant Analysis (sPLS-DA)—Factor-loading weights in component 1 are shown for the top 10 ranked parameters that predict active musculoskeletal involvements at baseline. (b) Forest plot-odds ratios (OR) with 95% confidence (by univariate logistic regression^{†}) of the predictors chosen by sPLS-DA with p value. (c) Regularised Random Forest (RRF)—Top 10 variables by mean decrease in Gini (ranked) represents the importance of each variable to predict the musculoskeletal involvements. (d) Multiple logistic regression^{†} to construct the final model to predict musculoskeletal involvements at screening, where variables were selected by random forest classification algorithm; with Area under the Receiver operator characteristic (ROC) curve (AUROC) of this final model to predict musculoskeletal involvements at screening.

FIG. 24—shows a network plot with Spearman's correlations to illustrate interrelationships between clinical and laboratory data. Correlations were tested among (1) (blue) Disease activities score [total and organ specific numerical-BILAG-2004^{†}, and SLEDAI-2K (systemic lupus erythematosus disease activity index 2000)] or cellular components related to disease activities (Lymph=Lymphocyte and platelets), (2) (orange) various subtypes, and subclasses of anti-dsDNA antibodies, CD-19, & C3, and (3) (maroon) cytokines including interferon, interferon scores and C-reactive protein (CRP). Correlations with only p value of ≤0.05 are shown. Thickness and colour of the lines show the strength of the Spearman correlation as shown in the legend.

FIG. 25—shows predictors of major clinical response irrespective of treatment at 52 weeks. (a) Sparse Partial Least Squares Discriminant Analysis (sPLS-DA)—Factor-loading weights in component 1 are shown for the top 5 (chosen by model optimisation) ranked parameters to predict response irrespective of treatment at 52 weeks. (b) Forest plot—odds ratios (OR) with 95% confidence (by univariate logistic regression^{†}) of the predictors chosen by sPLS-DA with p values. (c) Regularised Random Forest (RRF)—Top 10 variables by mean decrease in Gini (ranked) represents the importance of each variable to predict treatment response. (d) Multiple logistic regression^{†} to construct the final model to predict response at 52 weeks, where variables were selected by random forest classification algorithm; with Area under the Receiver operator characteristic (ROC) curve (AUROC) of this final model to predict response.

FIG. 26—shows that two clusters of patients in the BEAT-lupus trial were identified using the DBSCAN (Density-Based Spatial Clustering of Applications with Noise) approach. (a) Patients were clustered by using their antibody profile (subtypes and sub-classes of anti-dsDNA, immunoglobins (subtypes and sub-classes), cell counts (neutrophils, lymphocytes, monocyte, basophil, eosinophil, platelet, CD-19), C-reactive protein, ESR (erythrocyte sedimentation rate), disease activity scores (SLEDAI-2K and numerical-BILAG-2004^{†}), interleukins/IL (IL-6, IL10-, IL-12, IL-17), tumour necrosis factor-alpha, interferon (alpha and gamma), and interferon based scores (type I, type I A, and type I B) at screening. (b) Number of patients who responded to the treatment (Belimumab after rituximab=Bel, Placebo after rituximab=Plc) and response [renal responders (R) versus non-responders (NR)] across the two-clusters. NA (not applicable) indicates these patients left the trial before 52 weeks. (c-h) Variables that were significantly different (by Mann-Whitney's U test for all except for numerical-BILAG-2004 student's where a t-test was applied) across the two clusters with p-values provided.

FIG. 27—demonstrates serum IL-6 and IL-12 levels between screening and 52 weeks stratified by treatment and response. Changes in serum IL-6 from screening to 52 weeks stratified by—(a) treatment with belimumab or placebo (after rituximab), (b) responders and non-responders in the belimumab arm, and (c) responders and non-responders in the placebo arm. Changes in serum IL-12 from screening to 52 weeks stratified by—(d) treatment with belimumab or placebo (after rituximab), (e) responders and non-responders in the belimumab arm, and (f) responders and non-responders in the placebo arm. Longitudinal linear mixed-effect model was fitted with random effect of patient ID and fixed effect of treatment group intercepting with trial times and adjusted for screening value, age, gender, concomitant mycophenolate (yes or no), and prednisolone dose at respective time points to calculate expected difference at 24 and 52 weeks. Estimated mean with 95% confidence intervals and number of patients at each time points (n) are shown; p values at weeks 24 (for IgA2 only) and 52 are provided.

FIG. 28—lists the sequences referred to herein.

### EXAMPLES

### Materials and Methods

### Patients

BEAT-LUPUS was a 52 weeks phase IIb, multicentre, UK based (16 centres), randomised, double blind, placebo-controlled parallel group superiority clinical trial investigating efficacy of belimumab administered 4 to 8 weeks after the first infusion of B cell depletion therapy (rituximab) in patients with SLE (Shipa, M., et al Annals of Internal Medicine, Oct. 26 (2021)). Patients were between 18 and 75 years old fulfilling classification criteria for SLE (Gordon, C., et al., Rheumatology 57, e1-e45 (2017)), and was due to be treated with rituximab due to failure of conventional therapy according to NHS England guidelines and the British guidelines for the management of SLE in adults (NHS England Interim Clinical Commissioning Policy Statement: Rituximab for the treatment of Systemic Lupus Erythematosus in adults. (2013)).

52-patients were assigned to the BEAT-LUPUS randomised control trial (26 on each arm), of whom 44-patients (21 on belimumab, 23 on placebo) provided clinical data at 52 weeks. Table 4 presents baseline characteristics of these 44 patients. Baseline characteristics of the total 52 patients who were randomised can be found in the Table 1 of the published BEAT-LUPUS trial (Shipa, M., et al Annals of Internal Medicine, Oct. 26 (2021)). All the patients provided blood samples at 52 weeks, and 43-patients provided blood samples at 52-weeks.

A validation cohort was obtained from patients with SLE attending the UCLH SLE clinic. Demographics of this validation cohort are shown in Table 5.

### Elisa (Enzyme-Linked Immunosorbent Assay)

A commercially available ELISA kit was used to analyse IgG, IgM, IgA anti-dsDNA antibodies (Abnova, Taiwan). IgE, subclasses of IgG (IgG1, IgG2, IgG3, IgG4) and IgA (IgA1 and IgA2) anti-dsDNA antibodies were analysed by an in-house prepared ELISA as described. Briefly, 96-well maxisorb plates were precoated with 100 μl/well protamine sulphate (500 μl/in millipore water) at 4° C. for 45 min. After washing, plates were coated with dsDNA from calf thymus [10 μl/ml DNA (Sigma, St Louis, MO, USA)] overnight at 4° C. After the wells were blocked with 1% BSA in PBS, 10 μL of diluted plasma sample [1:100 in PBS (pH 7.4, NaCl concentration 75 mM/l, Tween 20 0.1%] was added and incubated for 2 hours at room temperature. Plates were washed and HRP-conjugated antihuman monoclonal—IgG subclass (mouse, ThermoFisher), -IgA subclass (rabbit, ThermoFisher), and -IgE (mouse, ThermoFisher) secondary antibodies in dilution buffer were added. Following an incubation for 30 min at room temperature, plates were washed again and 100 μL of tetramethylbenzidine (TMB) solution was applied to each well, incubated for 15-30 min, and then added with equal volume of stopping solution (2 M H2SO4) and read the optical density at 450 nm. Total serum IgG1 (Human IgG1 ELISA kit, Stratech, cat #ORB564278-BOR), IgG2 (Human IgG2 ELISA kit, Stratech, cat #ORB564279-BOR), IgG3 (Human IgG3 ELISA kit, Stratech, cat #ORB564280-BOR), IgA1 (Human IgA1 ELISA kit, Stratech, cat #ORB564273-BOR), and IgA2 (Human IgA1 ELISA kit, Stratech, cat #ORB564274-BOR) were determined by commercially available ELISA kits. Serum BAFF was also measured with commercially prepared kit (Human BAFF/BLyS/TNFSF13B DuoSet ELISA, R&D Systems, cat #DY 124-05).

### Quantification of Serum Cytokines

IL (interleukin)-6, IL-10, IL-12, IL-17, TNF (tumour necrosis factor)-α, IFN (Interferon)-α, IFN-γ were measured using a Quanterix Simoa assay according to the manufacturer's instructions.

### Flow Cytometry

Cryopreserved peripheral blood mononuclear cells (PBMC) were isolated by density gradient centrifugation over Ficoll (Merk) according to the manufacturer's instructions. Cells were stained with Live/Dead Blue (ThermoFisher Scientific) before staining for CD4 (CD4—AF488, BD Biosciences), CD3 (CD3—BUV395, BD Biosciences), CXCR5 (CXCR5—AF647, Biolegend), ICOS (ICOS—PE/Dazzle594, Biolegend) and PD-1 (PD-1—BV711, Biosciences) surface expression. To analyse the subclasses of plasmablasts, PBMC were stained with CD19 (CD19 BUV395, BD Biosciences), CD27 (CD27 V450, BD Biosciences) and CD38 (CD38 BUV737, BD Biosciences), together with intracellular anti-IgA1 (IgA1 FITC, Cambridge Bioscience) and anti-IgA2 (IgA2 AF647, Cambridge Bioscience). Immune cell phenotyping was performed using an LSR2 or Fortessa flow cytometer (BD Biosciences) and Diva software, version 9.0.0, and analyzed by FlowJo v10.7.1 (BD Bioscences).

### Definition of Clinical Response and Flare

Active renal disease defined as—BILAG-2004 index A or B in the renal domain at screening with a 24-hour urinary protein>500 mg/day, or urine protein-creatinine ratio (uPCR)>50 mg/mmol, or urine albumin-creatinine ratio (uACR)>50 mg/mmol at baseline, or active urinary sediment with uPCR>25 mg/mmol with active urinary sediment value (Davies, J. C., et al., RMD Open 6 (2020); Merrill, J. T., et al., Arthritis Rheum. 62, 222-233 (2010)). Active mucocutaneous disease defied as either A or B scores in BILAG-2004 mucocutaneous domain. Active musculoskeletal disease defied as either A or B scores in BILAG-2004 musculoskeletal domain. These definitions of active diseases were applicable for both the cohorts.

Major clinical response (MCR) was defined as reduction in BILAG-2004 index A or B scores to BILAG-2004 C (or D/E) in all domains, a reduction in steroid dose to ≤7.5 mg daily and a modified SLEDAI-2K score≤2 (without anti-dsDNA antibody component) (Trixy David et al., Arthritis Rheumatol. 73 (2021)).

Renal response defined as BILAG-2004 A or B in the renal domain at screening with a 24-hour urinary protein>500 mg/day, or urine protein-creatinine ratio (uPCR)>50 mg/mmol, or urine albumin-creatinine ratio (uACR)>50 mg/mmol at baseline, or active urinary sediment with either uPCR>25 mg/mmol, or uACR>25 mg/mmol and at 12 months-No BILAG-2004 A or B in the renal domain and a 24-hour urinary protein≤500 mg/day or urine protein-creatinine ratio (uPCR)≤50 mg/mmol, or urine albumin-creatinine ratio (uACR)≤50 mg/mmol at baseline, or no active urinary sediment with either uPCR≤25 mg/mmol or uACR≤25 mg/mmol and estimated Glomerular Filtration Rate (eGFR) using the MDRD (Modification of Diet in Renal Disease) formula≥60 mls/min/1.72 m² OR if eGFR≤60 mls/min at baseline, eGFR to not have fallen by ≥20% compared to the screening value (value (Davies, J. C., et al., RMD Open 6 (2020); Merrill, J. T., et al., Arthritis Rheum. 62, 222-233 (2010)).

Mucocutaneous and musculoskeletal response was defied as reduction in BILAG-2004 A or B scores to BILAG-2004 C (or D/E) in all domains, a reduction in steroid dose to ≤7.5 mg daily a SLEDAI-2K score≤2 (without anti-dsDNA antibody component).

### Statistical Analysis

Statistical analysis was performed using R software version 4·0·2 for Mac OS (R Foundation for Statistical Computing, Vienna, Austria). Two-sided p-values and 95% confidence intervals was reported for all statistical tests. A p-value threshold of 5% was used to reject the null hypothesis to ensure that the probability of a type I error does not exceed 5%.

For the demographic data were reported as mean (with standard deviation) or median (with interquartile range), depending on the data distribution. For continuous variables, comparison between 2-groups were analysed using either t-test (for parametric) or Mann-Whitney U test. As the data was high-dimensional, to select important variables supervised machine learning approaches were applied-sparse Partial Least Squares Discriminant Analysis (sPLS-DA) and Regularised Random Forest (RRF). Odds ratio (OR) with 95% confidence interval (95% CI) of the variables selected by sPLS-DA to predict outcome/organ involvement by univariable logistic regression and Mean decrease in Gini of the top 10 variables selected by RRF were report. Final model was constructed by using the variables selected by Boruta variable selection approach (Degenhardt, F., Seifert, S. & Szymczak, S., Brief. Bioinform. 20, 492-503 (2017)). For the longitudinal changes a linear mixed-effect model was fitted to estimate the mean change from randomisation to 52 weeks with fixed effect of treatment group or treatment response intercepting with trial times from randomisation to 52 weeks, random effect of within-patient, and adjusted for screening value, age, gender and concomitant mycophenolate (yes or no) and prednisolone dose at respective time. For the numerical BILAG-2004 changes through to 52 weeks, an unadjusted generalised linear mixed-effect model was fitted. For correlations non-parametric Spearman's rank correlation test was used. For clustering, K-means clustering was used where clusters were chosen by the number of clusters with Eigenvalue>1 from the principal component analysis (PCA). DBSCAN (Density-Based Spatial Clustering of Applications with Noise) when k-means clustering was fitted poorly.

### Example 1—Baseline Serum IgA2 Anti-dsDNA Antibody Levels Emerged as the Most Significant Predictor of Major Clinical Response at 52 Weeks to Treatment

The baseline (screening) and clinical demographic data from the BEAT-lupus trial (18) have been published, together with analysis of serum levels of IgG anti-dsDNA antibody, disease flares and adverse events during the 52 weeks of the trial. Clinical data were available for all the 52 patients who were randomised to the trial, however 44 of them provided clinical data at 52 weeks to define the clinical response (Number of available laboratory data were different for different parameters at each time point and are reported in the respective sections/figures). A major clinical response (MCR) (Trixy David, et al., Arthritis Rheumatol. 73 (2021)). was achieved in (10 responders, 11 non-responders) 47% of patients in the BEAT-lupus trial who received belimumab after rituximab compared to 34% (8 responders, 15 non-responders) in the placebo group (i.e. rituximab alone), added to standard of care, at 52 weeks. A model was constructed utilising a range of clinical and laboratory data (Table 1) to identify variables at baseline (screening) that could predict response at 52 weeks.

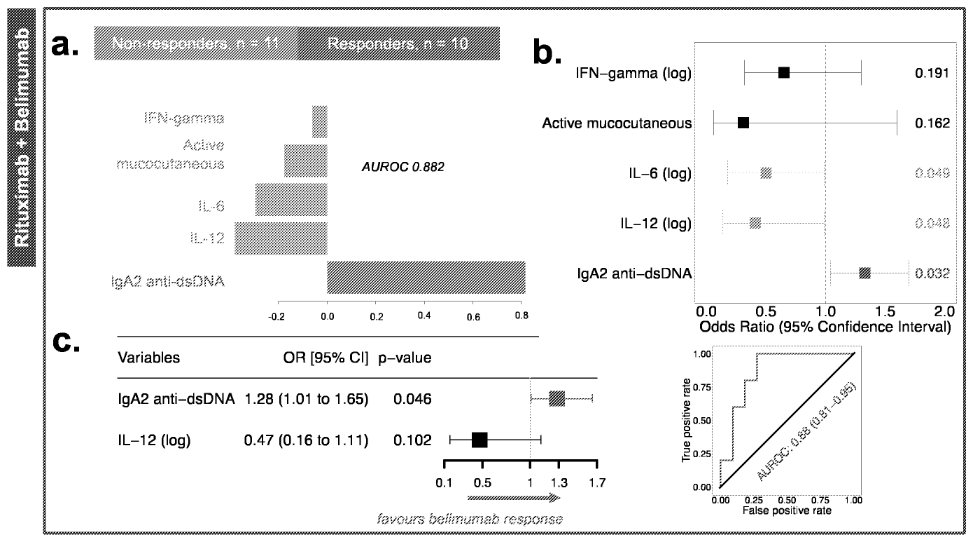

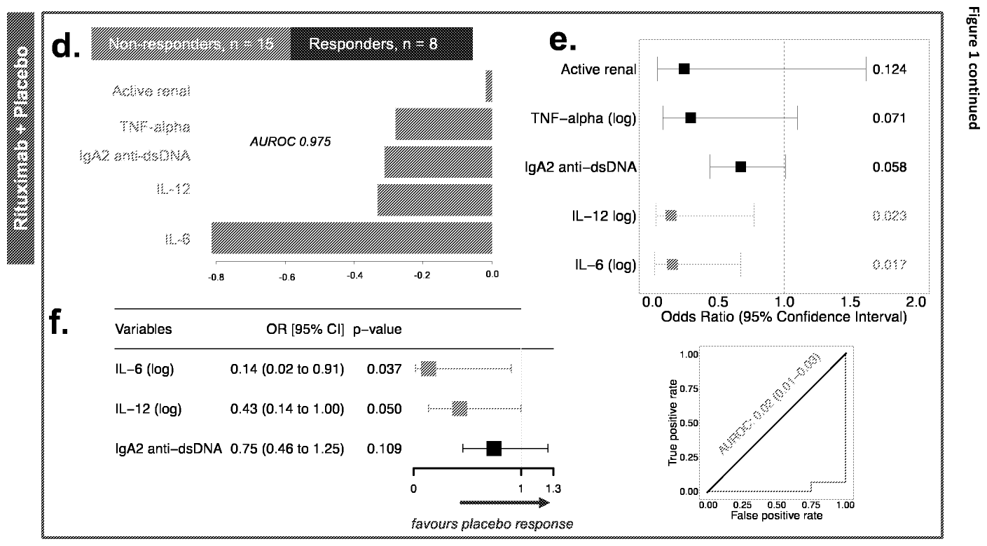

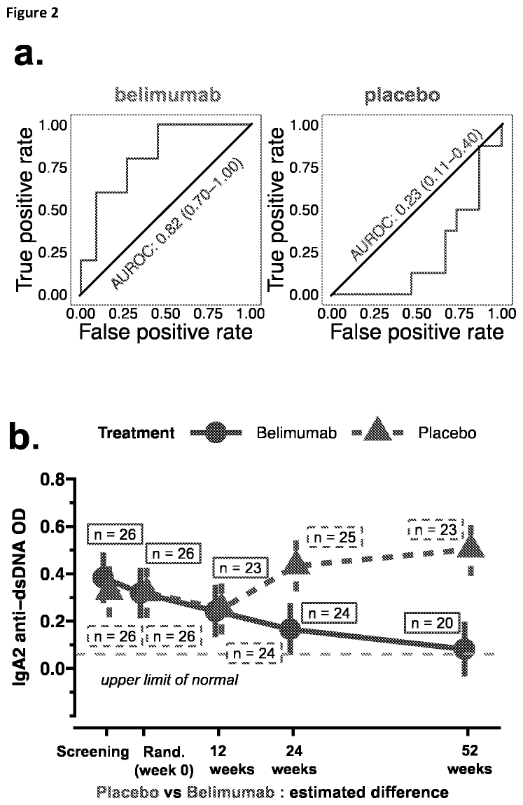

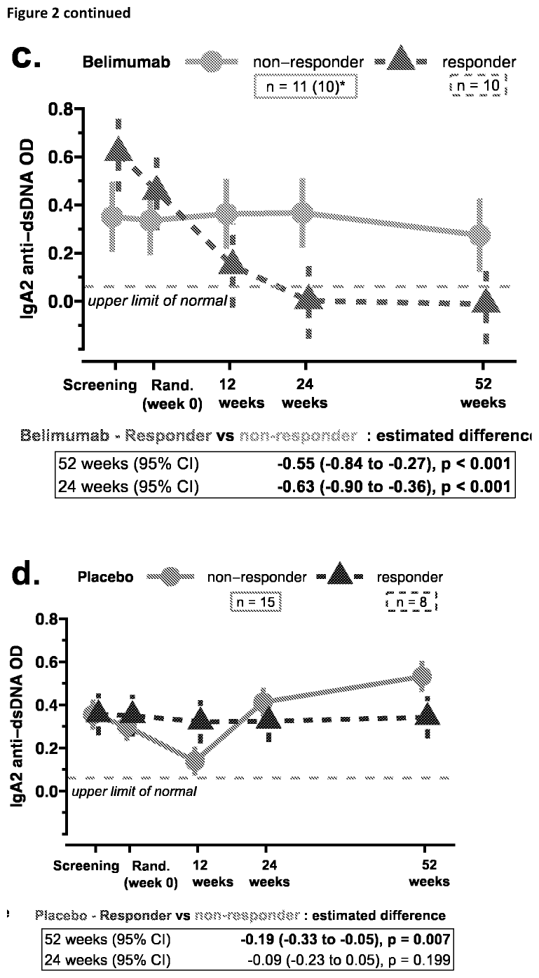

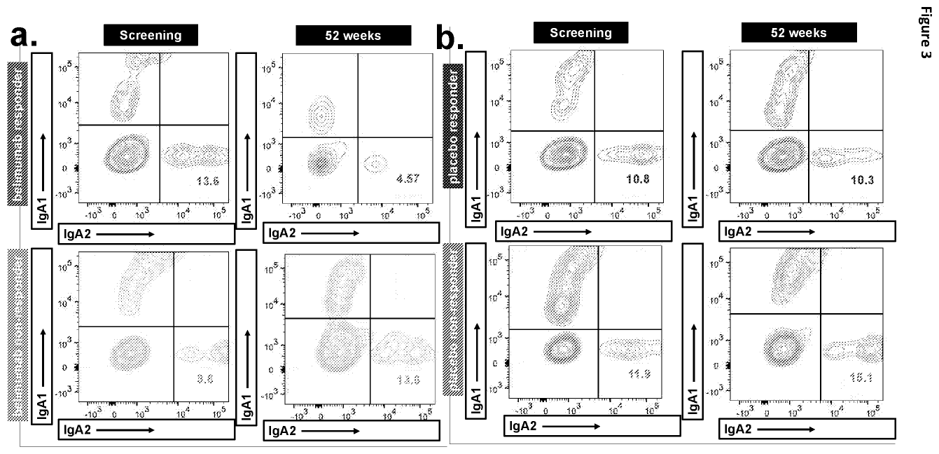

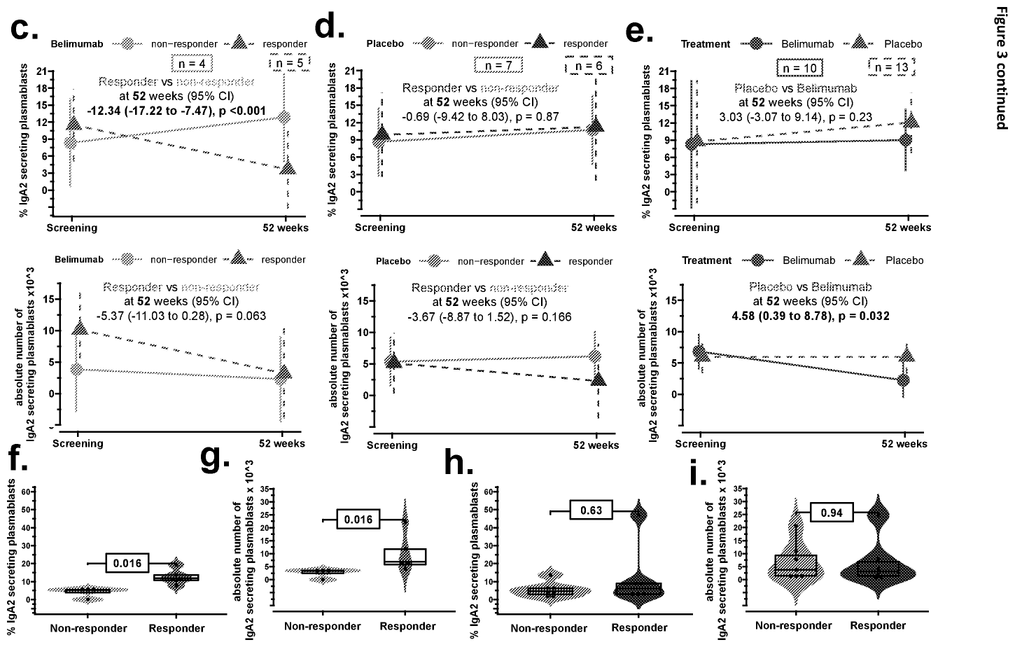

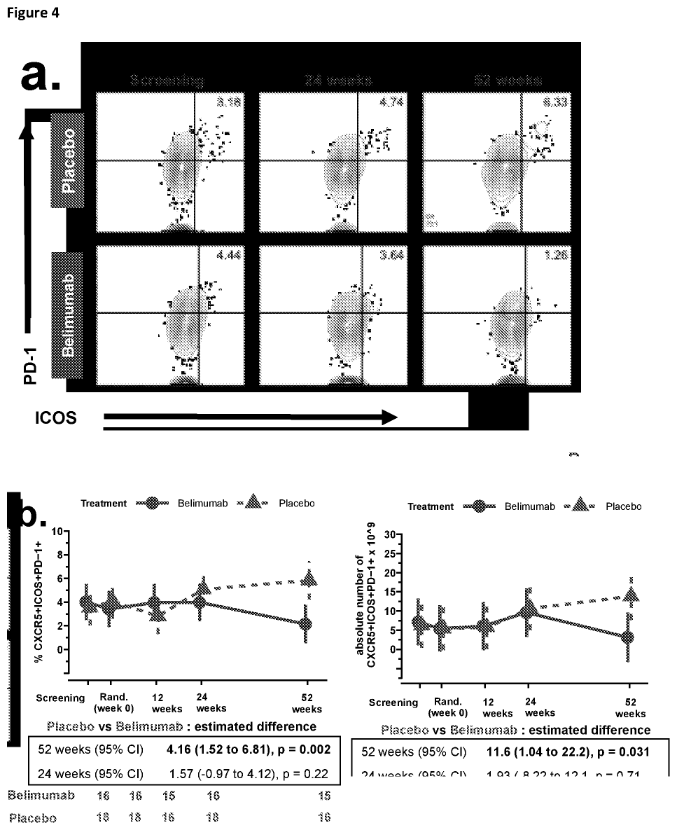

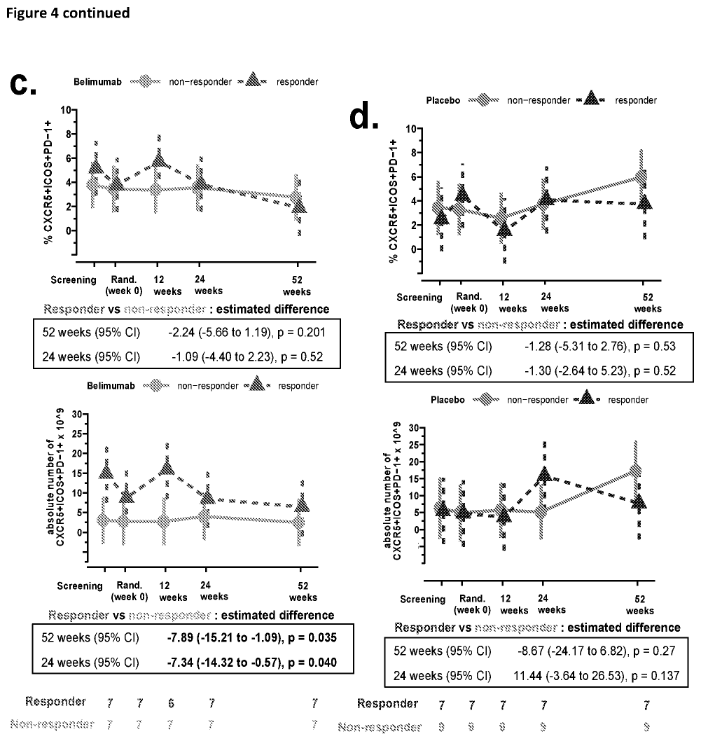

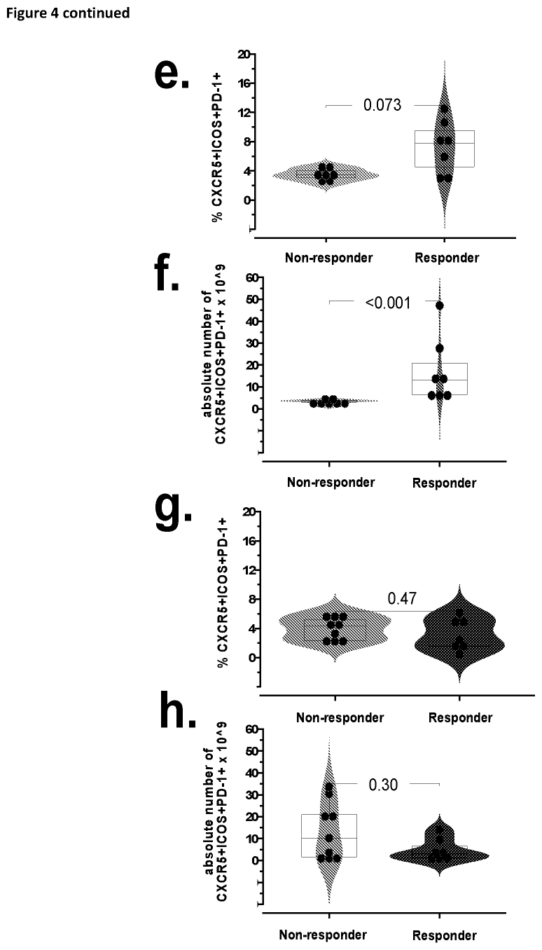

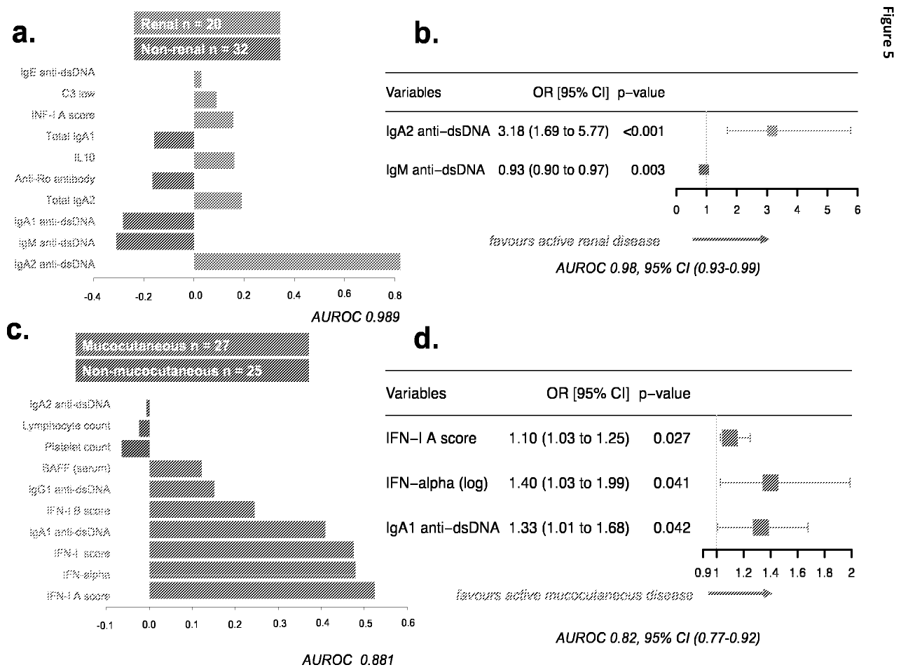

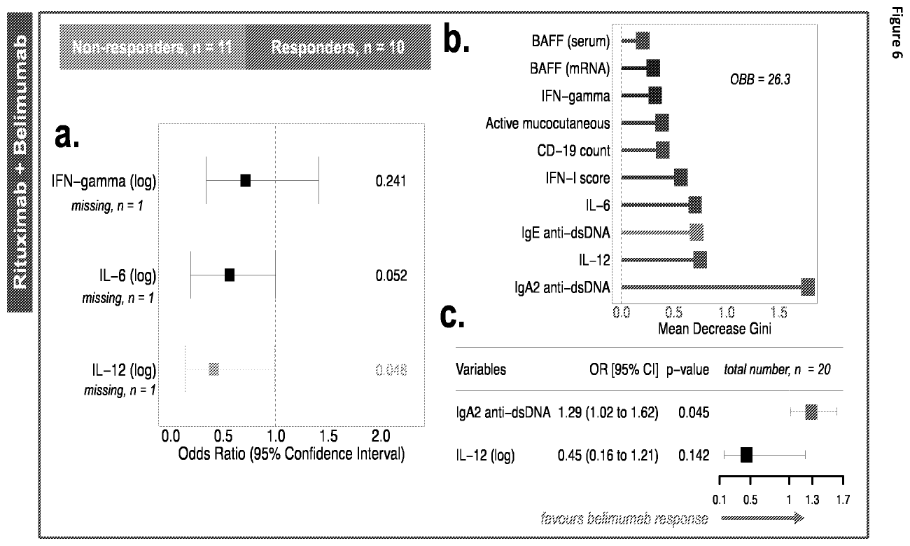

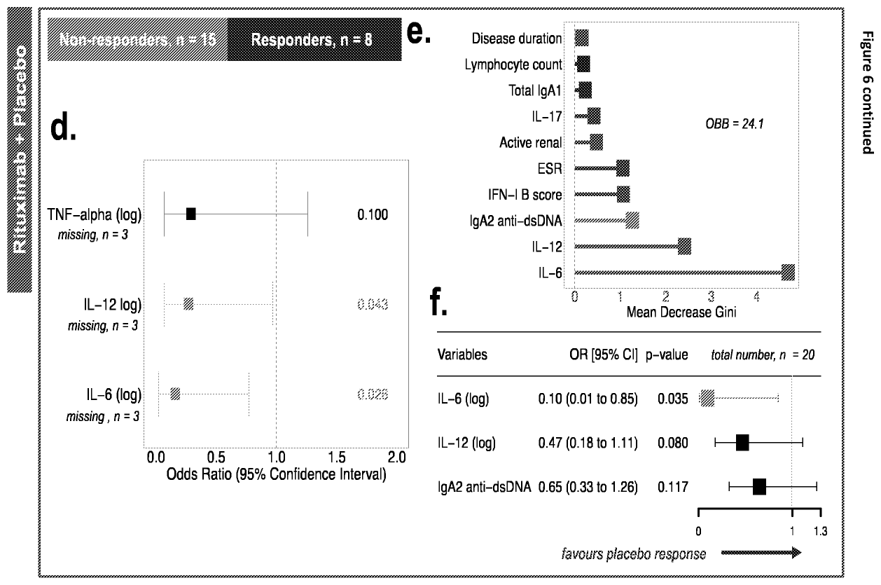

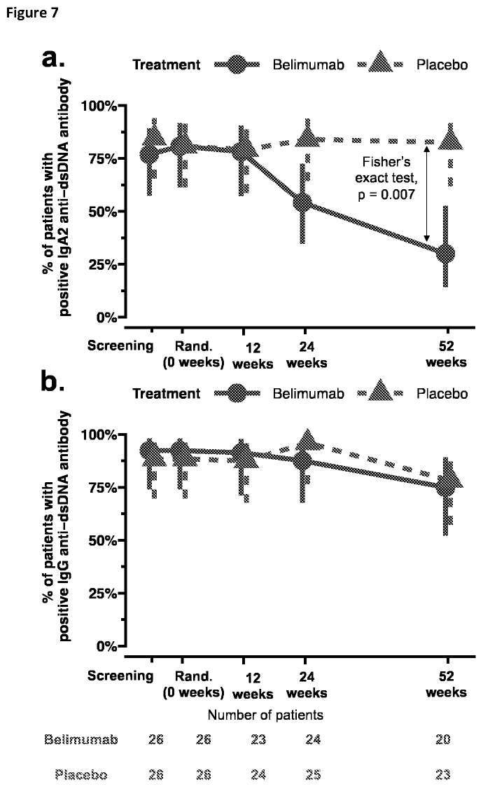

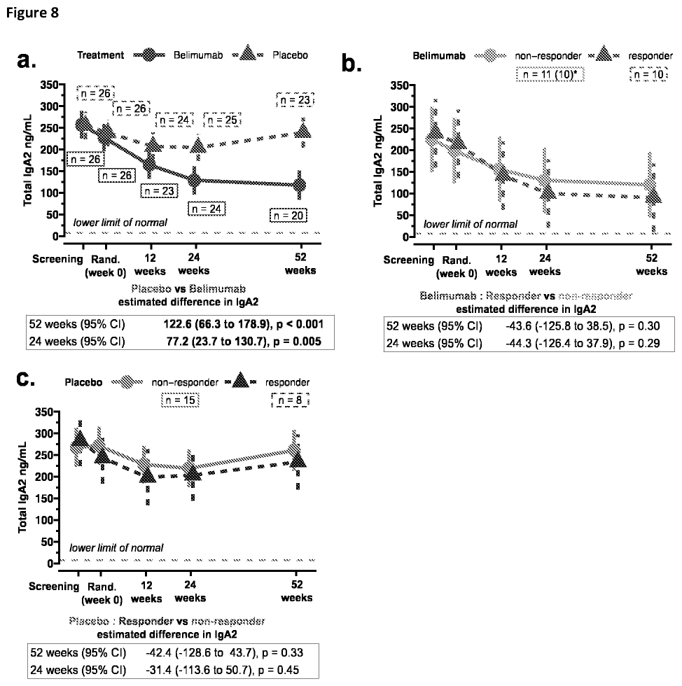

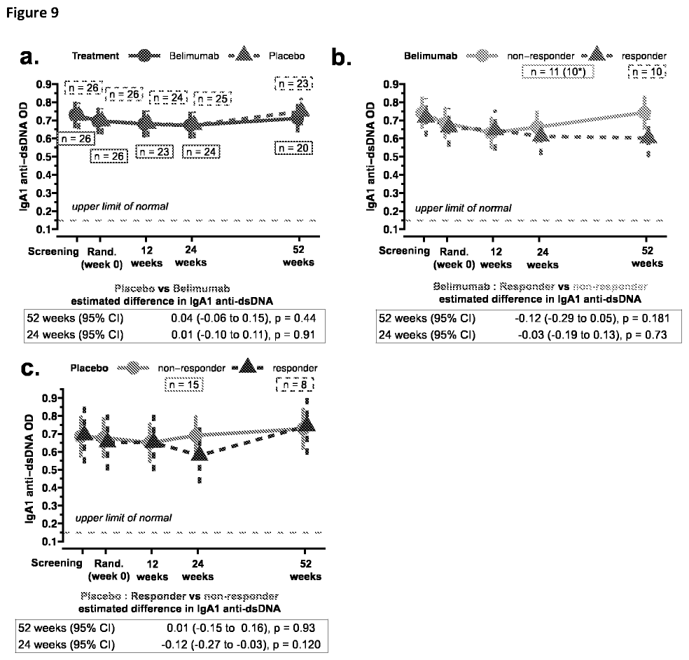

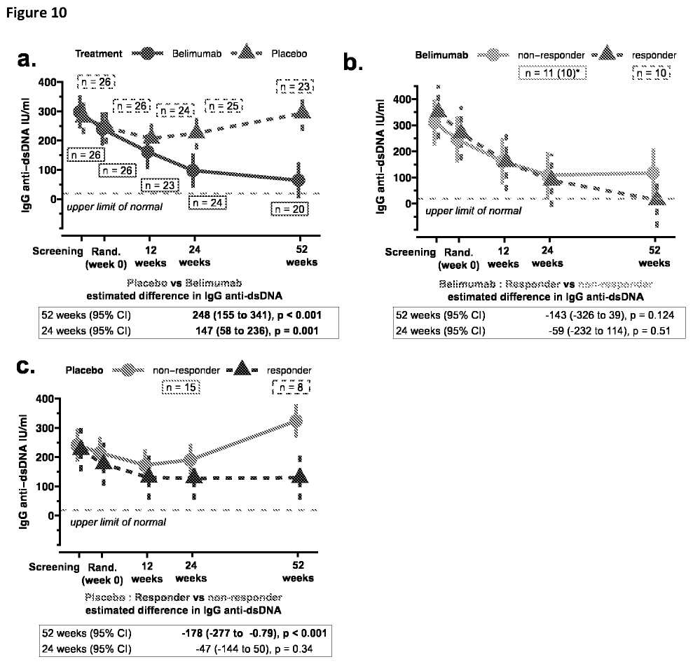

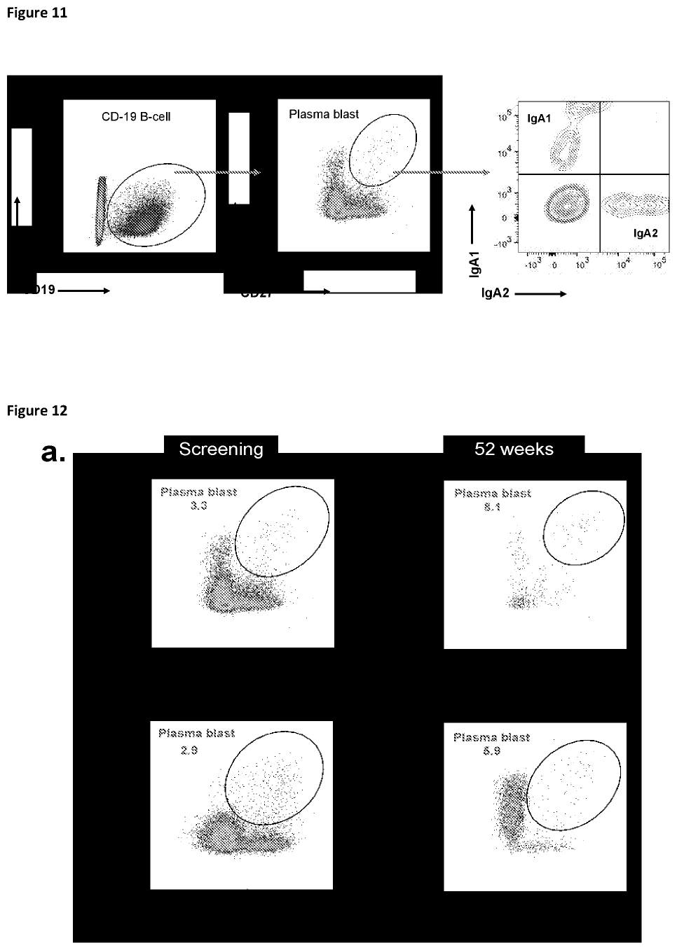

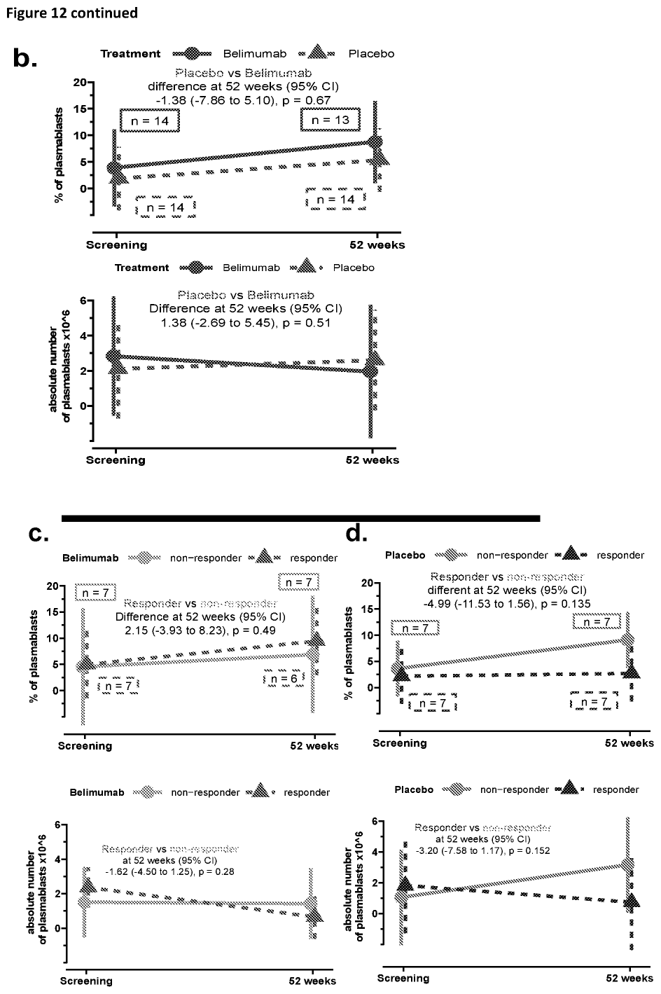

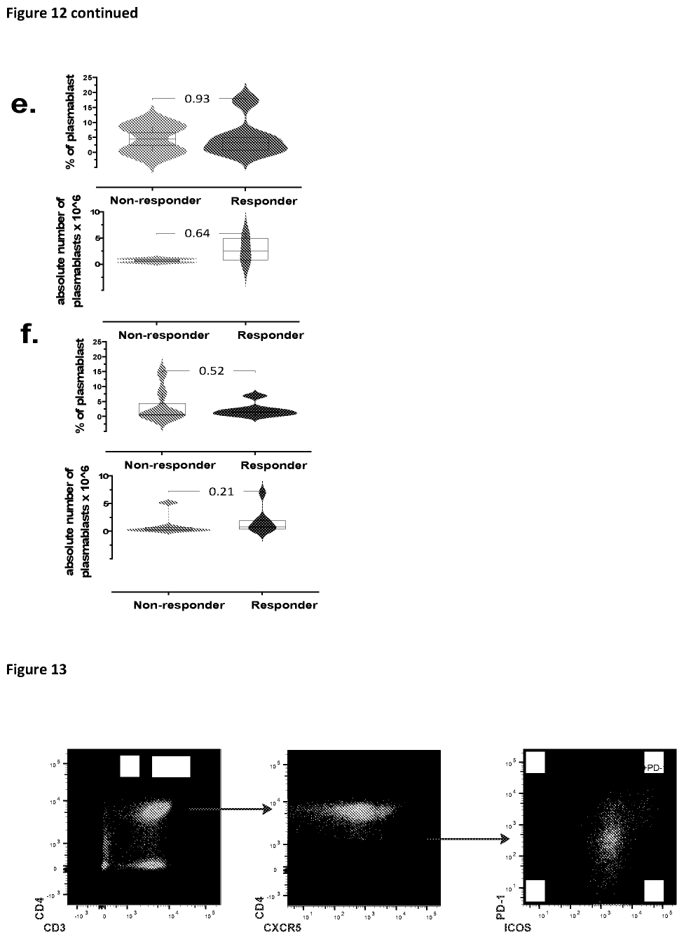

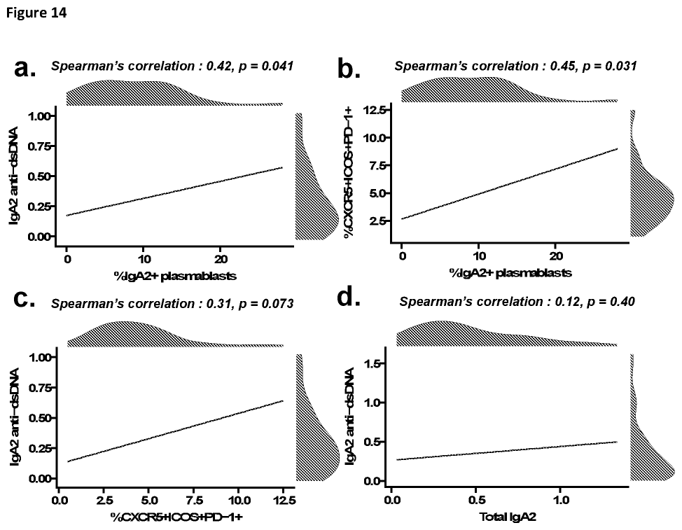

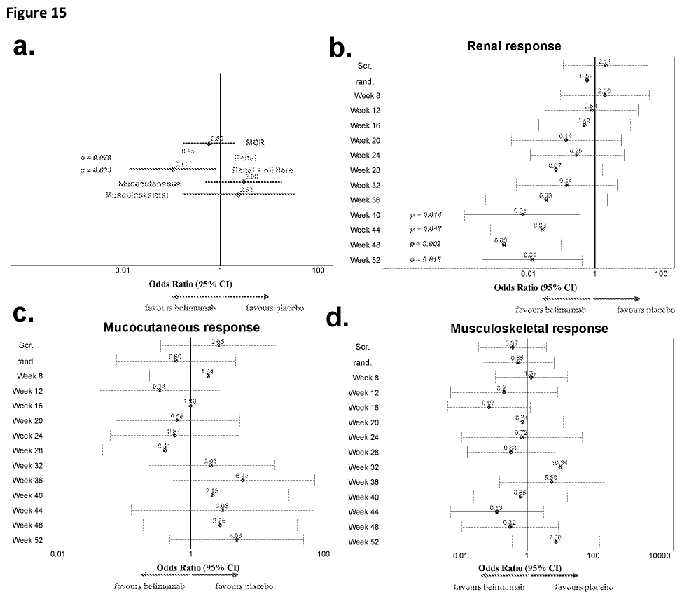

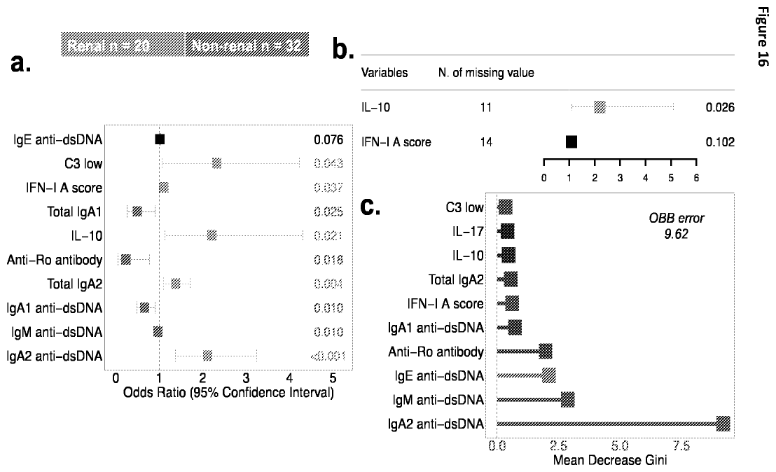

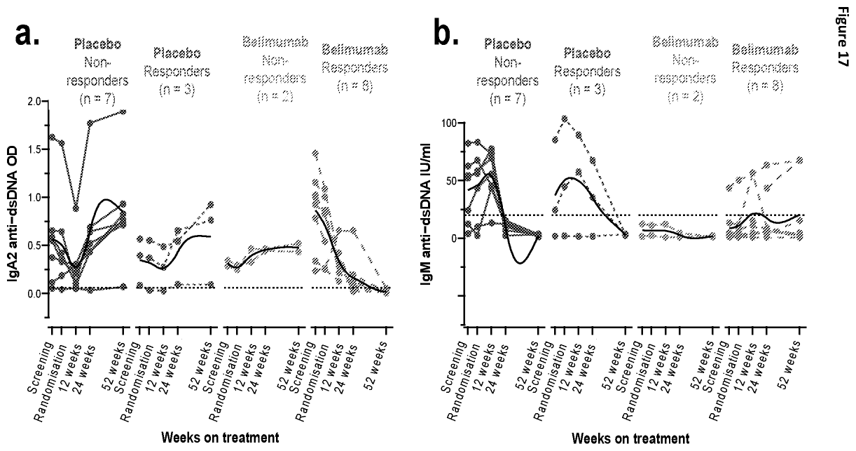

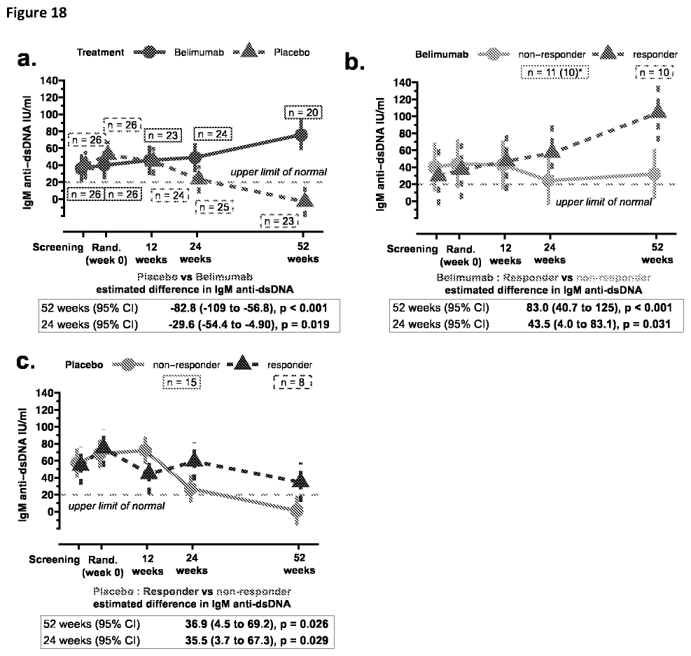

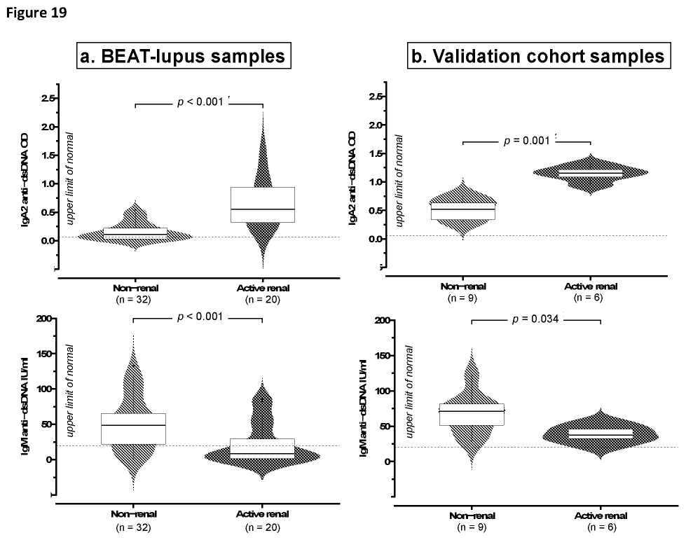

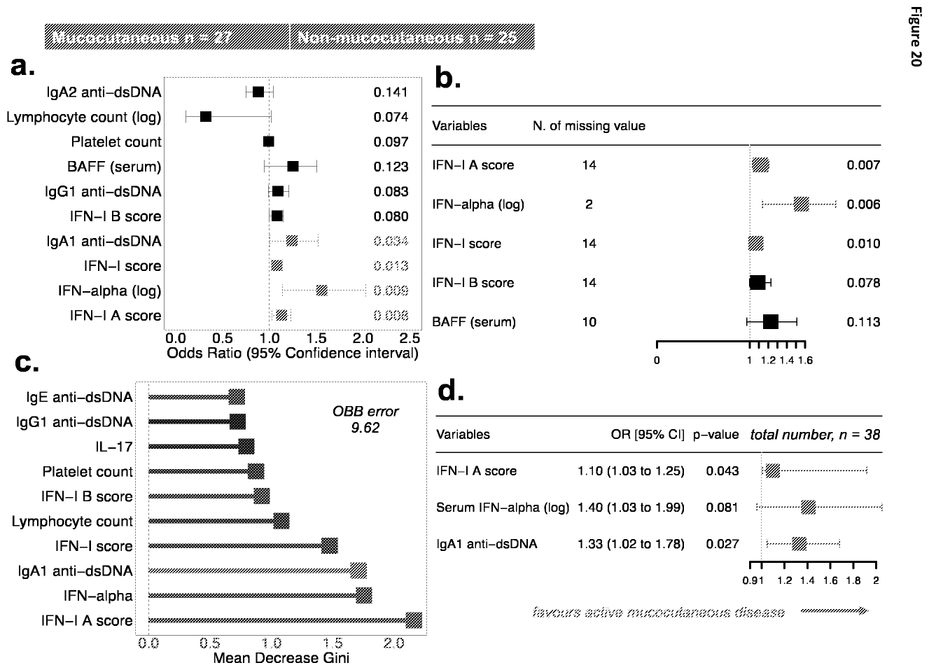

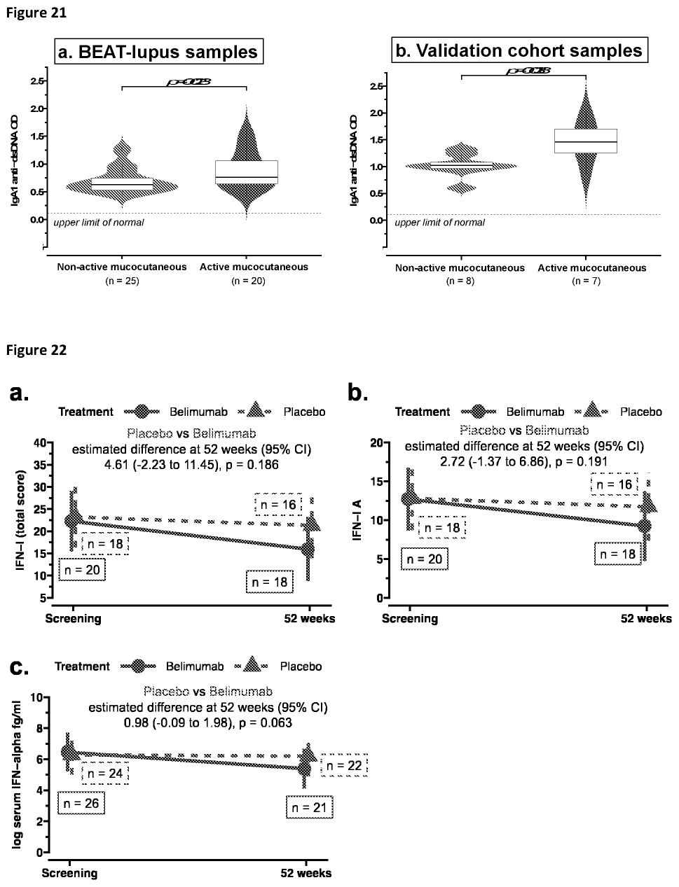

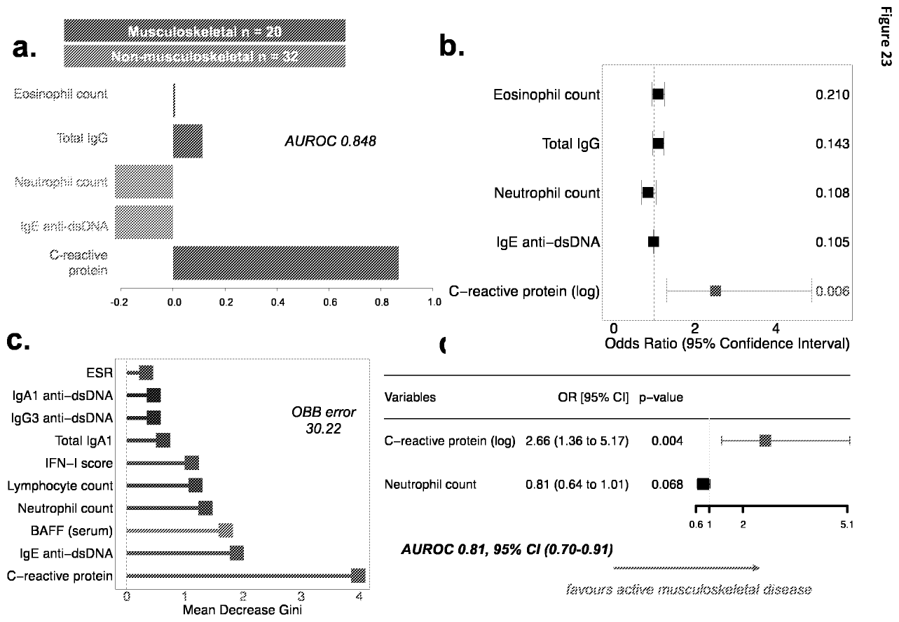

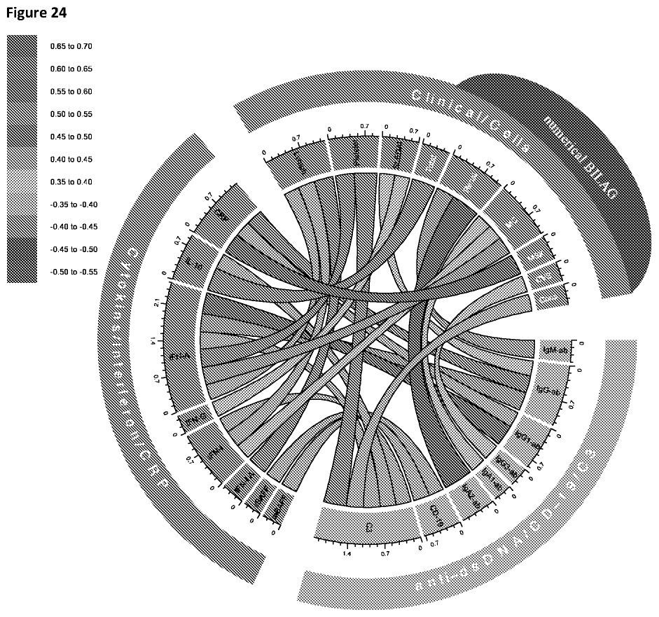

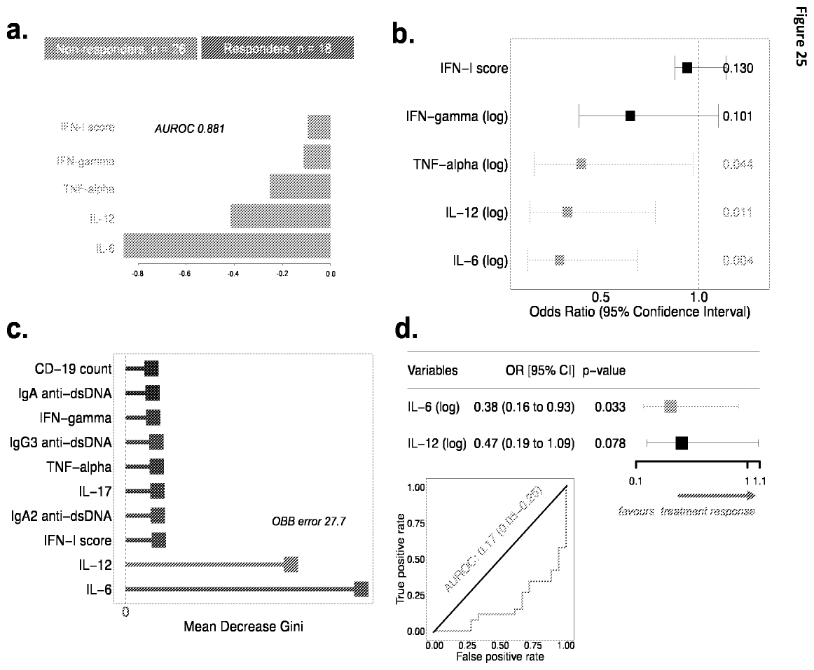

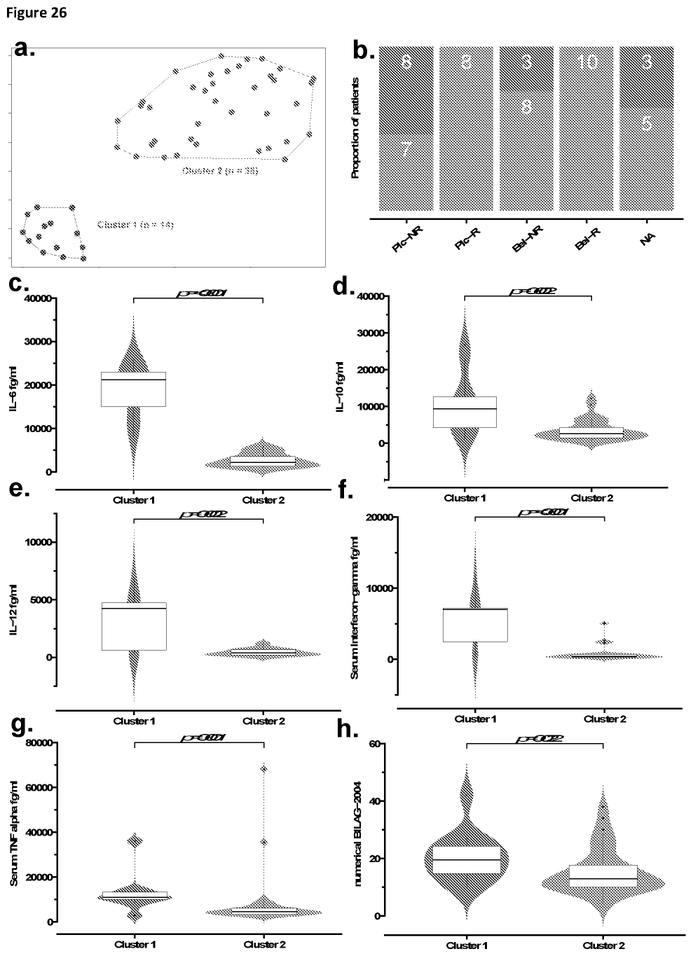

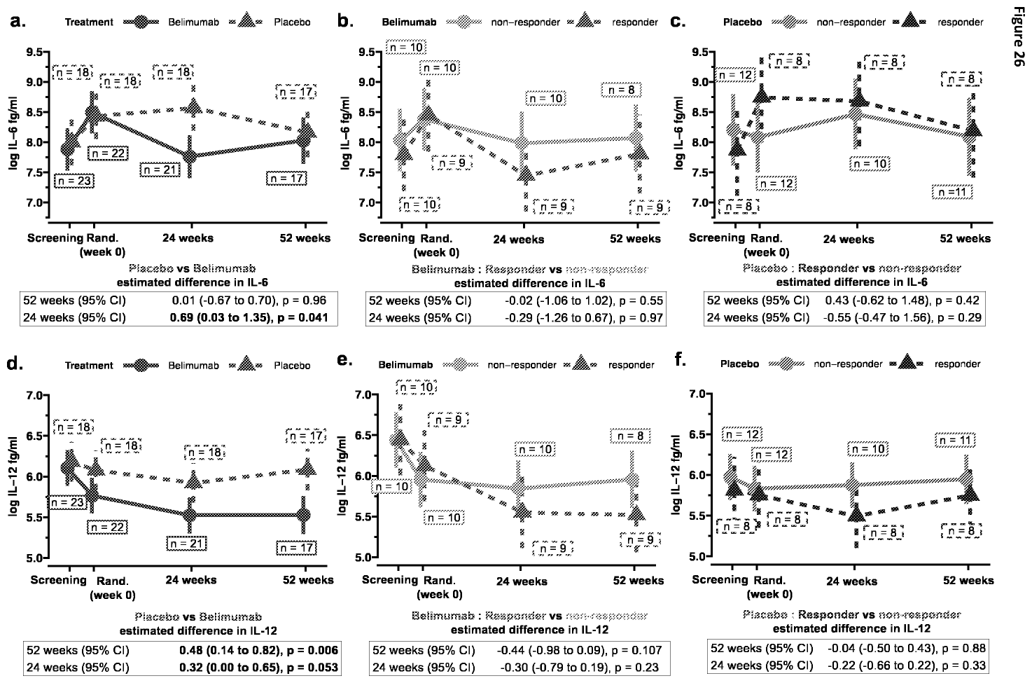

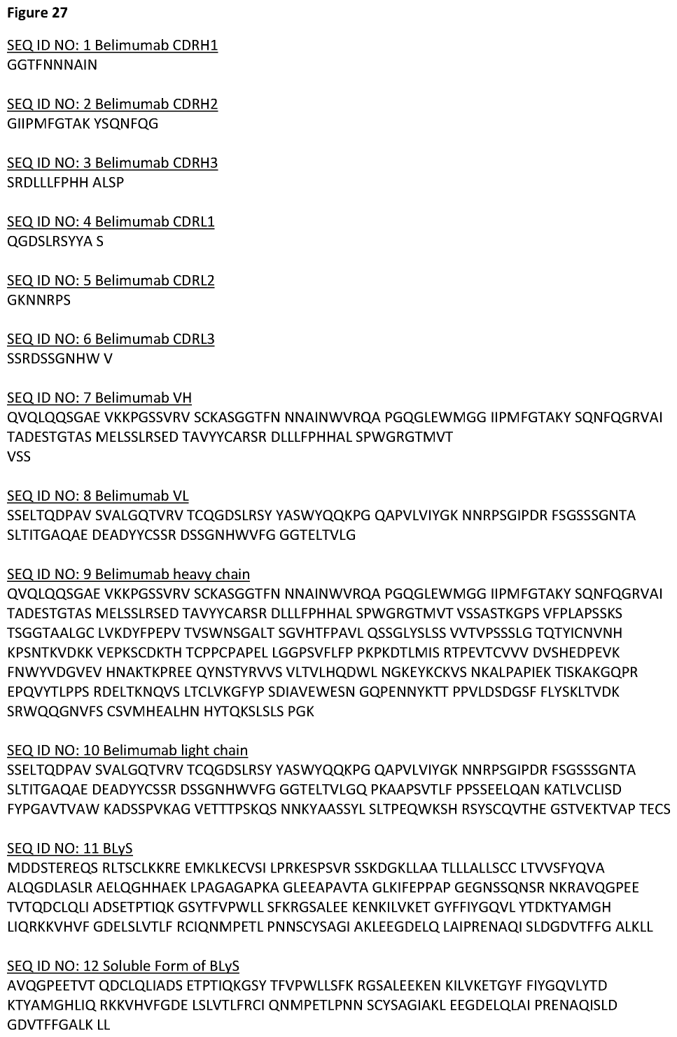

Out of a wide range of clinical and laboratory data, baseline serum IgA2 anti-dsDNA antibody levels emerged as the most important variable (selected by sparse partial least squares discriminant analysis, sPLS-DA) in predicting major clinical response (MCR) in belimumab treated patients (FIG. 1a), and yielded the only positive odds ratio/OR (1.33, p=0.032) of the top 5 variables identified (FIG. 1b). IL-12 and IL-6 were among the next important predictors associated with non-responder status (imputed ORs of univariate model are shown on FIG. 1b and complete case analysis in FIG. 6a). A sensitivity analysis using regularised random forest (RRF) confirmed serum IgA2 anti-dsDNA antibody levels as the most influential variable (by the mean decrease Gini score) to predict MCR at 52 weeks (FIG. 6b). Serum IgA2 anti-dsDNA antibody and IL-12 levels were selected by random forest classification algorithm (Kursa, M. B. & Rudnicki, W. R. Feature Selection with the Boruta Package. 2010 36, 13 (2010)) for the final multiple logistic regression model (FIG. 1c, complete Case analysis: FIG. 6c). Each increase by 0.1 in the optical density (OD) measured in the ELISA assay for serum IgA2 anti-dsDNA antibody levels in belimumab treated patients at screening yielded an OR of attaining MCR at 52 weeks of 1.28 (p=0.046) (FIG. 1c). Area under the receiver operator characteristic curve (AUROC) of this final model in predicting MCR was 0.88.

In contrast, all of the top 5 baseline variables selected by sPLS-DA from patients in the placebo arm of the trial were associated with an unfavourable outcome at 52 weeks (FIG. 1d). Serum IL-6 (log transformed) was the strongest negative predictor of attaining a MCR at 52 weeks (OR 0.15, p=0.017), followed by serum IL-12 and IgA2 anti-dsDNA antibody levels (FIG. 1e). Variables with missing value showed similar ORs by complete case analysis (FIG. 6d). RRF was then adopted to verify the sensitivity of these variables in predicting MCR (FIG. 6e), and the importance of IL-6 was confirmed. In the final multivariate model (FIG. 1f, Complete Case analysis: FIG. 6f), the OR of serum IL-6 as a negative predictor of MCR at 52 weeks was 0.14 (p=0.037). The AUROC of the final model in predicting MCR in placebo treated patients was 0.02.

In summary, placebo controlled clinical trials using targeted therapies offer a unique opportunity to progress the understanding of disease pathogenesis and molecular stratification. Using clinical and laboratory data from patients in the BEAT-lupus trial, serum IgA2 anti-DNA antibodies was identified as the only positive predictor of response to belimumab after rituximab in SLE. IgA2 outperformed other anti-DNA antibody isotypes, including IgG anti-dsDNA antibody.

### Example 2—Belimumab after Rituximab Reduced Serum IgA2 Anti-dsDNA Antibody Levels Only in Patients Who Responded to Therapy

Serum IgA2 anti-dsDNA antibody levels alone reliably predicted MCR in patients treated with belimumab (AUROC 0.82) and negatively predicted a MCR in the placebo arm (AUROC 0.23) (FIG. 2a). The optimal cut-point for IgA2 anti-dsDNA antibody to predict response in belimumab arm was 0.19 OD, with a sensitivity of 1.00, and a specificity of 0.55. Belimumab significantly reduced serum IgA2 anti-dsDNA antibody levels compared to placebo at 24 (p<0.001) and 52 weeks (p<0.001) (FIG. 2b). Scrum IgA2 anti-dsDNA antibody levels decreased only in those patients treated with belimumab that achieved a MCR, falling by 92% from baseline (p<0.001) (FIG. 2c). There was no such reduction from baseline to 52 weeks in serum IgA2 anti-DNA antibody levels in the placebo group irrespective of response (FIG. 2d), though non-responders in the placebo arm increased their serum IgA2 anti-dsDNA antibody levels from baseline to 52 weeks. At baseline 77% (20 out of 26) and 85% (22 out of 26) of patients were positive for serum IgA2 anti-dsDNA antibody levels (IgA2 anti-dsDNA antibody positivity was defined as above 3 standard deviation of the mean value of health donors, n=15) in belimumab and placebo arms, respectively; which reduced to 30% (6 out of 20) at 52 weeks in the belimumab group, but remained unchanged between the two time points in those patients receiving placebo (FIG. 7a), (fisher exact test comparing normalisation of serum IgA2 anti-dsDNA antibody levels between the belimumab and placebo arms at 52 weeks=p<0.001). In contrast, the percentage of patients who remained IgG anti-dsDNA antibody positive at 52 weeks were similar between the belimumab and placebo group (FIG. 7b). Total serum IgA2 levels (FIG. 8a) were decreased by 54% in belimumab treated patients. However, in contrast to IgA2 anti-dsDNA antibody levels, total IgA2 levels did not differ in patients according to clinical response either in the belimumab or placebo group (FIG. 8b, c). In contrast to serum IgA2 anti-dsDNA antibody levels, serum IgA1 anti-dsDNA antibody levels were not affected by either combination belimumab and rituximab, or rituximab alone (FIG. 9a-c). A significant reduction in serum IgG anti-dsDNA antibody levels in patients treated with belimumab compared to placebo after rituximab (FIG. 19a) was shown previously (Shipa, M., et al. Annals of Internal Medicine, Oct. 26 (2021)). But no significant difference in serum IgG anti-dsDNA antibody levels was observed at 24 or 52 weeks between belimumab responders and non-responders (FIG. 10b). However, serum IgG anti-dsDNA antibody levels were lower in responders compared to non-responders in the placebo group at 52 weeks (p<0.001) (FIG. 10c).

### Example 3—Elevated Baseline Circulating IgA2 Secreting Plasmablast Numbers Predict Clinical Response and were Reduced by Belimumab after Rituximab

The dynamics of intracellular IgA2 secreting peripheral blood plasmablasts was analysed, using flow cytometry, from a subset of patients in the BEAT-lupus trial (where samples were available, belimumab n=10, placebo n=13) divided according to response at 52 weeks (Gating strategy shown in FIG. 11). There was a significant reduction in the percentage of IgA2 secreting plasmablasts in the peripheral blood of patients treated with belimumab who achieved a MCR in contrast to patients that did not respond (p<0.001) (FIG. 3a, c). In contrast, there was no change in patients treated with placebo after rituximab irrespective of response (FIG. 3b, d). The absolute number of peripheral blood IgA2 secreting plasmablasts reduced in the belimumab group at 52 weeks compared to the placebo arm (p=0.032) (FIG. 3e). The absolute number and percentage of IgA2 secreting plasmablast were higher at screening in the belimumab treated patients who achieved a MCR at 52 weeks (FIG. 3 f, g). In contrast, there was no difference in the number (or frequency) of IgA2 secreting plasmablasts in the placebo treated patients divided according to response (FIG. 3 h, i). Compared to IgA2 secreting plasmablasts, the frequency and number of total plasmablasts (Gating strategy shown in FIG. 11) was unchanged between the belimumab and placebo group at 52 weeks (FIG. 12 a, b) and their response (FIG. 12 c, d). Baseline total plasmablast percentage or absolute number were not different between responders and non-responders (FIG. 12 c, f).

### Example 4—Increased Baseline T-Follicular Helper Cell Number Predict Clinical Response to Belimumab after Rituximab

Circulating T follicular helper cells (Tfh) were analysed as key drivers of plasma cell differentiation. Tfh were defined as CD4⁺CXCR5⁺ICOS⁺PDI⁺ using flow cytometry (gating strategy shown in FIG. 13). Representative flow cytometry analysis showed a significant reduction in the frequency (p=0.003) and number (p=0.03) of Tfh in belimumab treated patients from baseline to 52 weeks compared to placebo (FIG. 4a, b). A significant reduction was also observed in the number of Tfh in belimumab treated patients who attained a MCR (p=0.025) compared to those not reaching this response threshold (FIG. 4c) whereas there was no difference in responders and non-responders in the placebo arm of the trial (FIG. 4d). The absolute number and percentage of Tfh were higher at baseline in patients who achieved a MCR at 52 weeks in the belimumab arm (FIG. 4 e, f). The baseline frequency or absolute number of Tfh was not different for responders compared to non-responders in the placebo treated group (FIG. 4 g, h). Significant correlations were noted between the screening values of serum IgA2 ant-dsDNA antibody levels, percentages of IgA2 secreting plasmablasts and Tfh (FIG. 14a-d).

The data points to inhibition of IgA2 anti-dsDNA antibody production, associated with changes in Tfh and IgA2 secreting plasmablasts, as a key mechanism of action of belimumab after rituximab in SLE. Enumeration of circulating plasmablasts secreting IgA1 and IgA2 and Tfh supports serological analysis, and demonstrates that inhibition of BAFF targets systemic T-B cell interactions and that IgA2 autoantibodies are secreted systemically, possibly having migrated from the colon. There were also changes in circulating Tfh which stimulate the differentiation of B cells into plasmablasts in the context of lupus. The frequency and number of circulating cells with a Tfh like phenotype were reduced by belimumab after rituximab, whereas Tfh were unchanged after rituximab alone. Tfh frequency has been shown to correlate with plasmablasts, IgG anti-DNA antibodies and disease activity in a proportion of patients with SLE, in particular glomerulonephritis.

### Example 5—Active Renal and Mucocutaneous Disease were Associated with Raised Serum IgA2 Anti-dsDNA and IgA1 Anti-dsDNA Antibody Levels Respectively

It has previously been shown that patients treated with belimumab were more likely to achieve a renal response and/or no new renal flare [by BILAG-2004 (British Isles lupus assessment group-2004) A/B in renal domain] compared to placebo after rituximab (Floris et al., Autoimmunity Reviews 15, 656-663 (2016)). In contrast, the favourable effect of belimumab was not observed for mucocutaneous or musculoskeletal disease (FIG. 15a). The improvement in renal disease with belimumab was clearly observed from week 40 through to 52 weeks (FIG. 15b) but not for mucocutaneous or musculoskeletal involvement (FIG. 15c, d).

Clinical and laboratory data those were applied to construct prediction model (Table 1), were also used to construct a model to predict active organ specific disease at screening (52 patients has clinical data at screening, number of missing laboratory data are reported in the Table 2).

Serum IgA2 anti-dsDNA antibody levels was the most important variable associated with active renal disease at baseline (FIG. 5a). OR (by univariate regression) of active renal disease of 2.11 (p<0.001) for each 0.1 OD increase in IgA2 anti-dsDNA antibody (FIG. 16a). Complete case analysis for those parameters with missing values are shown in FIG. 16b. By RRF, the importance of serum IgA2 anti-dsDNA antibody levels stood out from all the variables analysed (FIG. 16c). Serum IgA2 and IgM anti-dsDNA antibody levels were selected by the random forest classification algorithm (FIG. 5b) and fitted in to the final model (AUROC 0.98). In multivariate analysis, for each 0.1 OD increase in IgA2 anti-dsDNA antibody, the risk of active renal disease increased with OR of by 3.18 (p<0.001), whereas a rise in IgM anti-dsDNA antibody by 1 IU/ml, reduced the risk of renal disease with an OR of 0.93 (p=0.003). Reduction in serum IgA2 anti-dsDNA antibody levels was also seen in belimumab treated patients who showed a renal response at 52 weeks (FIG. 17a). An increasing trend was observed in IgM anti-dsDNA antibody for the belimumab treated renal patients (FIG. 17b) and overall, serum IgM anti-dsDNA antibody levels were increased in belimumab treated patients at 24 (p=0.019) and 52 (p<0.001) weeks compared to placebo (FIG. 18a) which was accompanied higher IgM anti-dsDNA antibody in responders compared to non-responders in both treatment-arm (FIG. 18b, c). To validate the association between serum IgA2 anti-dsDNA antibody levels and renal involvement, serum levels of IgA2 and IgM anti-dsDNA antibodies were analysed in an independent cohort. Serum IgA2 anti-dsDNA antibody levels were significantly higher in renal disease, whereas IgM anti-dsDNA antibody levels were lower, in renal disease in both patient cohorts (FIG. 19a, b).

It was next sought to identify a molecular signature that associated with active mucocutaneous disease in the BEAT-lupus samples. Interferon (IFN) induced expression signatures, and the derived type 1 total (IFN-I score) and type 1A scores (IFN-I A score), serum IFN-α levels, and IgA1 anti-dsDNA antibody levels were the dominating variables predictive of active mucocutaneous disease (FIG. 5a) by sPLS-DA and their ORs (by univariable regression) were statistically significant both imputed (FIG. 20a) and complete case analysis (FIG. 20b). By RRF, the importance of these variables stood out from all the variables analysed (FIG. 20c). IFN-1A expression signature, serum IFN-α, and IgA1 anti-dsDNA antibodies were selected by the random forest classification algorithm and were fitted in the final model (FIG. 5d). In the final multivariate model, for each unit increase in IFN-I A score (FIG. 5d), the risk of active mucocutaneous increased by OR of 1.10 (p=0.027). For serum IFN-α the OR (log transformed) was 1.40 (p=0.041). Each 0.1 OD increase in serum IgA1 anti-dsDNA antibody levels increased risk by an OR of 1.33 (p=0.042). Multivariate model with complete case analysis revealed similar result (FIG. 20d). To validate the association between serum IgA1 anti-dsDNA antibody levels and mucocutaneous involvement, serum levels of IgA1 anti-dsDNA antibody was analysed in an independent cohort. Serum IgA1 anti-dsDNA antibody levels were significantly higher in mucocutaneous disease (FIG. 21a, b). Belimumab did not significantly suppress serum IgA1 anti-dsDNA antibody, interferon signatures or serum levels (FIG. 22a-c) levels compared to placebo.

CRP (C-reactive protein) was the most important determinant to predict active musculoskeletal involvement in lupus, by sPLS-DA and RRF (FIG. 23a-c). CRP and neutrophil were selected by the random forest classification algorithm and were fitted in the final model (FIG. 23d). For serum CRP the OR (log transformed) was 2.66 (p=0.004).

Numerical BILAG-2004 (Cresswell, L., et al., Rheumatology (Oxford) 48, 1548-1552 (2009)) was also correlated with various serum anti-dsDNA antibodies, C3, cytokines, BAFF, IFN, and IFN based transcriptional scores (FIG. 24). Serum IgA2 anti-dsDNA antibody levels showed the strongest positive correlation with numerical BILAG-2004 in the renal domain at screening with spearman's correlation coefficient of 0.68 (95% CI 0.48 to 0.79, p<0.001). The mucocutaneous numerical BILAG-2004 score was positively correlated with serum IFN-α (0.43, 95% CI 0.36 to 0.64, p=0.008), IFN-I (0.40, 95% CI 0.33 to 0.62, p=0.009), and IgA1 anti-dsDNA antibody (0.39, 95% CI 0.31 to 0.02, p=0.017). The musculoskeletal domain was strongly correlated with serum CRP, with spearman's correlation coefficient 0.46 (95% CI 0.37 to 0.67, p=0.007).

### Example 6—Serum IL-6 was Associated with a Poor Response to B Cell Targeted Therapy

In view of the substantial proportion of patients that did not achieve a MCR irrespective of their treatment, it was sought to identify the key variables that were associated with a failure to reach a MCR at 52 weeks in the whole trial cohort (FIG. 25a-d). Serum IL-6 at baseline was associated with an unfavourable response (OR of MCR 0.38, p=0.033) and this result was not affected by complete case analysis (Table 3). 2 patient groups were identified by DBSCAN (Density-Based Spatial Clustering of Applications with Noise) clustering (FIG. 26a). Strikingly, in cluster 1 none of the 14 patients achieved a MCR (FIG. 26b) to either belimumab or placebo, whereas in cluster 2, 15 patients out of 33 patients did not achieve a MCR (fisher exact test p=0.001). Patients in cluster 1 had a disproportionately higher baseline serum IgA1 anti-dsDNA antibody, IL-6, IL-12, IFN-γ and TNF-α levels (FIG. 26c-g) compared to cluster 2. These patients also had higher disease activity at screening as evident by their numerical BILAG-2004 (Isenberg, D. A., et al. BILAG 2004, Rheumatology 44, 902-906 (2005)), as shown is FIG. 26h.

As raised serum IL-6 levels were associated with significantly poorer response to B cell targeted therapy (as shown above) at 52 weeks, the longitudinal changes in IL-6 in belimumab and placebo treated patients was further analysed (FIG. 27). In both arms, IL-6 remained unchanged from screening to 52 weeks (FIG. 27a). Minimal changes in IL-6 through to 52 weeks occurred when stratified by response (FIG. 27b, c). In contrast, despite its unfavourable effects on MCR, IL-12 level was associated with greater reduction (FIG. 27d) at 52 weeks (p=0.006). IL-12 changes were similar between MCR and non-MCR in both placebo and belimumab group (FIG. 27e, f).

The following examples are prophetic in nature.

### Example 7—Samples from SLE Patients in Alternative Clinical Studies Wherein Belimumab has been Used Both as a Monotherapy or in Differing Dosage Regimens in Combination with Rituximab Will be Tested for IgA2 Anti-dsDNA

Patients will be recruited from different cohorts, for example healthy patients and those who are diagnosed with SLE. Samples will be collected from the patients at different time intervals and IgA2 autoantibody, such as IgA2 anti-dsDNA autoantibody, levels measured for each.

The initial treatment may be with belimumab or rituximab or placebo. Patients with elevated IgA2 anti-dsDNA levels will then be treated with belimumab. Patients will then be monitored for those that respond to treatment.

For example a retrospective analysis will be conducted on remaining serum samples from the clinical trial titled BLISS-BELIEVE (NCT03312907), to evaluate whether baseline levels or change from baseline in post-treatment levels of serum IgA2 anti-dsDNA antibodies can predict clinical response to belimumab monotherapy and/or to belimumab therapy after a single cycle of rituximab (which may be administered during belimumab therapy), in SLE patients. Additional analysis may include the retrospective testing or remaining samples from belimumab studies in patients with Lupus Nephritis (BLISS-LN: NCT01639339) and in paediatric patients with childhood-onset SLE (PLUTO: NCT01649765).

TABLE 1
Baseline variables used in the prediction model

| | |
|---|---|
| Demographics | Age $, gender††, race (Caucasian, black, |
| | and others) ††, active smoking†† |
| Disease status | Disease duration at screening $, previous |
| | rituximab††, SLEDAI-2K‡ (systemic lupus |
| | erythematosus disease activity index 2000) |
| Active organ | Renal††, mucocutaneous††, |
| involvement††† | musculoskeletal†† |
| Concomitant | Prednisolone††, Prednisolone dose ‡, |
| treatment | mycophenolate††, azathioprine††, |
| | methotrexate†† |
| Biochemical and | Lymphocyte‡‡, neutrophil‡, eosinophil‡‡, |
| immunological | basophil‡‡, monocyte‡‡, platelet‡, CD19 |
| profile | count‡‡; c-reactive protein (CRP) ‡‡, |
| | erythrocyte sedimentation rate (ESR) ‡, |
| | complement C3 (normal or low) †† |
| Serum | IgG anti-SM††, IgG anti-Ro††, IgG anti- |
| autoantibodies | LA††, IgG Anti-RNP††, IgG anti-dsDNA‡, |
| | IgM anti-dsDNA‡, IgA anti-dsDNA‡, IgE |
| | anti-dsDNA‡‡‡, IgG1 anti-dsDNA‡‡‡, |
| | IgG2 anti-dsDNA‡‡‡, IgG3 anti- |
| | dsDNA‡‡‡, IgA1 anti-dsDNA‡‡‡, IgA2 |
| | anti-dsDNA‡‡‡, (IgG4 anti-dsDNA |
| | antibody was also measured but the values |
| | were not above those from healthy donors) |
| Serum total | IgG‡, IgG1‡, IgG2‡, IgG3‡, IgM‡, IgA‡, |
| Immunoglobulins (Ig) | IgA1‡, IgA2‡ |
| Serum Cytokines | BAFF‡ (B cell activating factor) (2), IL |
| | (interleukin)-6‡‡ (4), IL-10‡‡ (4), IL-12‡‡ |
| | (4), IL-17‡‡ (4), TNF (tumour necrosis |
| | factor)-α ^{‡‡} (4), IFN (Interferon)-α ^{‡‡} (1), |
| | IFN-γ ^{‡‡} (4) |
| Interferon score and | BAFF‡ (7), Type I IFN score (IFN-I) ‡ (7), |
| BAFF RNA expression | Type I A IFN score (IFN-I A) ‡ (7), Type B |
| | IFN total score (IFN-I B) ‡ (7), |

†Screening refers to the first screening visit before rituximab, randomisation (week 0) occurred 4-8 weeks after screening.
^{¶} Only patients who provided clinical data at 52 weeks, so that the response can be determined, were included.
The number of missing values at screening for each variable are provided (in italics) after the respective variable, where applicable.
††Categorial variable.
†††Active renal disease defined as - BILAG-2004 (British Isles lupus assessment group - 2004) index A or B in the renal domain at screening with a 24-hour urinary protein >500 mg/day, or urine protein-creatinine ratio (uPCR) >50 mg/mmol, or urine albumin-creatinine ratio (uACR) >50 mg/mmol at baseline, or active urinary sediment with uPCR >25 mg/mmol with active urinary sediment. Active mucocutaneous disease defined as either A or B scores in BILAG-2004 mucocutaneous domain. Active musculoskeletal disease defined as either A or B scores in BILAG-2004 musculoskeletal domain.
Unit changes for the continuous variables for the logistic regression:
$ per 1 year change,
‡per 1 unit change,
^{‡‡} log-transformed,
‡‡‡per 0.1 OD (optical density) change

TABLE 2
Baseline variables used in the prediction
model for active organ involvement

| | |
|---|---|
| Demographics | Age $, gender††, race (Caucasian, black, |
| | and others) ††, active smoking†† |
| Disease status | Disease duration at screening $, previous |
| | rituximab††, SLEDAI-2K‡ (systemic lupus |
| | erythematosus disease activity index 2000) |
| Concomitant treatment | Prednisolone††, Prednisolone dose ‡, |
| | mycophenolate††, azathioprinett††, |
| | methotrexate†† |
| Biochemical and | Lymphocyte‡‡, neutrophils‡, eosinophil‡‡, |
| immunological profile | basophil‡‡, monocyte‡‡, platelet‡, CD19 |
| | count‡‡; c-reactive protein (CRP) ‡‡, |
| | erythrocyte sedimentation rate (ESR) ‡, |
| | complement C3 (normal or low) †† |
| Serum autoantibodies | IgG anti-SM††, IgG anti-Ro††, IgG anti- |
| | LA††, IgG Anti-RNP††, IgG anti-dsDNA‡, |
| | IgM anti-dsDNA‡, IgA anti-dsDNA‡, IgE |
| | anti-dsDNA‡‡‡, IgG1 anti-dsDNA‡‡‡, |
| | IgG2 anti-dsDNA‡‡‡, IgG3 anti- |
| | dsDNA‡‡‡, IgAl anti-dsDNA‡‡‡, IgA2 |
| | anti-dsDNA‡‡‡,(IgG4 anti-dsDNA |
| | antibody was also measured but the values |
| | were not above those from healthy donors) |
| Serum total | IgG‡, IgG1‡, IgG2‡, IgG3‡, IgM‡, IgA‡, |
| Immunoglobulins (Ig) | IgA1‡, IgA2‡ |
| Serum Cytokines | BAFF‡ (B cell activating factor) (2), IL |
| | (interleukin)-6‡‡ (4), IL-10‡‡ (4), IL-12‡ |
| | (4), IL-17‡‡ (4), TNF (tumour necrosis |
| | factor)-α ^{‡‡} (4), IFN (Interferon)-α ^{‡‡} (1), |
| | IFN-γ ^{‡‡} (4) |
| Interferon score and BAFF | BAFF‡ (7), Type I IFN score (IFN-I) ‡ (7), |
| RNA expression | Type I A IFN score (IFN-I A) ‡ (7), Type B |
| | IFN total score (IFN-I B) ‡ (7), |

^{‡}Screening refers to the first screening visit before rituximab, randomisation (week 0) occurred 4-8 weeks after screening.
^{¶} Active renal disease defined as - BILAG-2004 (British Isles lupus assessment group - 2004) index A or B in the renal domain at screening with a 24-hour urinary protein >500 mg/day, or urine protein-creatinine ratio (uPCR) >50 mg/mmol, or urine albumin-creatinine ratio (uACR) >50 mg/mmol at baseline, or active urinary sediment with uPCR >25 mg/mmol with active urinary sediment. Active mucocutaneous disease defined as either A or B scores in BILAG-2004 mucocutaneous domain. Active musculoskeletal disease defied as either A or B scores in BILAG-2004 musculoskeletal domain.
^{††} Categorial variable.
^{#} Numerical-BILAG-2004 - where BILAG-2004 (British Isles lupus assessment group - 2004) A score = 12, B score = 6, C score = 1, D/E score = 0.
Unit changes for the continuous variables for the logistic regression:
^{$} per 1 year change,
^{‡} per 1 unit change,
^{‡‡} log-transformed,
^{‡‡‡} per 0.1 OD (optical density) change

TABLE 3
Logistic regression of the top 5-parameters to predict responsef
irrespective of treatment at 52 weeks (complete case analysis)

| | Number | | | | |
|---|---|---|---|---|---|
| | of total | Odds | | Odds | |
| | patients/ | ratio†† | | ratio†† | |
| | numbers of | (95% CI) | | (95% CI) | |
| | missing | Univariate | | Univariate | |
| Parameters | values | model | p-value | model | p-value |
| IL - 6 (log) | 40/4 | 0.30 (0.12 | 0.008 | 0.40 (0.15 | 0.047 |
| | | to 0.73) | | to 0.97) | |
| IL -12 (log) | 40/4 | 0.37 (0.16 | 0.022 | 0.57 (0.22 | 0.141 |
| | | to 0.87) | | to 1.17) | |
| TNF - alpha | 40/4 | 0.42 (0.16 | 0.069 | | |
| | | to 1.07) | | | |
| Serum IFN - | 40/4 | 0.66 (0.38 | 0.155 | | |
| gamma | | to 1.17) | | | |
| IFN-I score | 37/7 | 0.94 (0.88 | 0.081 | | |
| | | to 1.01) | | | |

^{†} Major clinical response - reduction in BILAG-2004 (British Isles lupus assessment group - 2004) index A or B scores to BILAG-2004 C (or D/E) in all domains, a reduction in steroid dose to ≤7.5 mg daily and a modified SLEDAI-2K (systemic lupus erythematosus disease activity index 2000) score ≤2 (without anti-dsDNA antibody component).
††Unit changes for the continuous variables used in the logistic regression are shown in table 1.
IFN = Interferon, IFN-I score = Type 1 interferon score, IL = interleukin, TNF = Tumour necrosis factor.

TABLE 4
Baseline demographics and disease characteristics of the patients
in the BEAT-lupus trial who provided clinical data at 52 weeks.

| | Belimumab | | Placebo | |
|---|---|---|---|---|
| | (n = 21) | | (n =23) | |
| Age, years ^{‡} | 39.5 | (12.1) | 42.1 | (10.5) |
| Sex - no. of patients (%) | | | | |
| Female | 17 | (81%) | 21 | (91%) |
| Ethnicity - no. of patients (%) ^{§} | | | | |
| White | 13 | (62%) | 16 | (70%) |
| Black | 3 | (14%) | 2 | (9%) |
| South Asian | 2 | (9.5%) | 1 | (4%) |
| Chinese | 1 | (5%) | 1 | (4%) |
| Other | 2 | (9.5%) | 3 | (13%) |
| Disease duration at screening^{††}, years | | | | |
| mean (SD) | 11.5 | (9.1) | 8.9 | (7.4) |
| median (IQR) | 10.3 | (4.5-17.7) | 6.2 | (3.5-14.3) |
| Previous rituximab - no. of patients (%) | 4 | (19%) | 7 | (30%) |
| Previous rituximab within 2 years from | 2 | (10%) | 3 | (13%) |
| screening - no. of patients (%) | | | | |
| Concomitant immunosuppressant or prednisolone | | | | |
| at screening, no. of patients (%) | | | | |
| Mycophenolate | 15 | (71%) | 13 | (57%) |
| Azathioprine | 2 | (9.5%) | 2 | (9%) |
| Methotrexate | 3 | (14%) | 2 | (9%) |
| Prednisolone | 18 | (86%) | 21 | (91%) |
| Receiving concomitant immunosuppressant or | 21 | (100%) | 23 | (100%) |
| prednisolone | | | | |
| Concomitant hydroxychloroquine at screening, | 17 | (81%) | 20 | (87%) |
| no. of patients (%) | | | | |
| Average daily prednisolone dose at screening, | | | | |
| mg/day | | | | |
| mean (SD) | 12.9 | (9.8) | 15.1 | (10.2) |
| median (IQR) | 9 | (5-18.8) | 13 | (8-20) |
| Patients taking ≥7.5 mg/day prednisolone, | 10 | (48%) | 16 | (70%) |
| no. of patients (%) at screening | | | | |
| Patients taking ≥10 mg/day prednisolone at | 9 | (43%) | 14 | (61%) |
| screening, no. of patients (%) at screening | | | | |
| Organ involvement (either BILAG-2004 A/B | | | | |
| system scores) at screening, no. of | | | | |
| patients (%) | | | | |
| Constitutional | 1 | (5%) | 2 | (9%) |
| Cardiorespiratory | 4 | (19%) | 6 | (26%) |
| Mucocutaneous | 10 | (48%) | 12 | (52%) |
| Musculoskeletal | 8 | (38%) | 8 | (35%) |
| Neuro-psychiatric | 0 | (0%) | 1 | (4%) |
| Ophthalmic | 0 | (0%) | 0 | (0%) |
| Gastro-intestinal | 0 | (0%) | 0 | (0%) |
| Renal | 10 | (48%) | 8 | (35%) |
| Haematological | 1 | (5%) | 0 | (0%) |
| BILAG-2004 system score at screening, no. | | | | |
| of patients (%) | | | | |
| ≥1 BILAG-2004 A | 6 | (29%) | 12 | (52%) |
| ≥1 BILAG-2004 A or 2 BILAG-2004 B | 13 | (62%) | 15 | (65%) |
| Numerical BILAG-2004^{¶} at screening | | | | |
| mean (SD) | 12.4 | (6.4) | 14.6 | (8.3) |
| median (IQR) | 12 | (7-14) | 14 | (7-16) |
| SLEDAI-2K at screening | | | | |
| mean (SD) | 10.9 | (3.9) | 11.3 | (4.8) |
| median (IQR) | 12 | (8-12) | 12 | (8-14) |
| Positive IgG anti-dsDNA antibody at | 19 | (90%) | 20 | (87%) |
| screening, no. of patients (%) | | | | |
| IgG anti-dsDNA antibody level at | | | | |
| screening, IU/ml | | | | |
| mean (SD) | 299 | (301) | 227 | (253) |
| median (IQR) | 165 | (97-318) | 161 | (60-254) |
| Low complement C3 at screening, no. | 12 | (57%) | 11 | (48%) |
| of patients (%) | | | | |
| Erythrocyte sedimentation rate (ESR) | | | | |
| at screening, | | | | |
| mean (SD) | 23.4 | (16.5) | 19.6 | (15.1) |
| median (IQR) | 20.5 | (12-28.2) | 16 | (10.2-22.8) |
| Lymphocyte count at screening, 10⁹/L | | | | |
| mean (SD) | 0.94 | (0.53) | 0.83 | (0.45) |
| median (IQR) | 0.78 | (0.58-1.28) | 0.78 | (0.50-0.95) |
| Platelet count at screening, 10⁹/L | | | | |
| mean (SD) | 273 | (97) | 273 | (107) |
| median (IQR) | 254 | (223-337) | 270 | (176-330) |
| Creatinine at screening (only renal | | | | |
| BILAG-2004 A or B), ml/min/1.73 m² | | | | |
| mean (SD) | 60.8 | (16.3) | 66.8 | (20.9) |
| median (IQR) | 56 | (49-72.3) | 71 | (53.2-83) |
| eGFR at screening (only renal | | | | |
| BILAG-2004 A or B), ml/min/1.73 m² | | | | |
| mean (SD) | 87.8 | (6.6) | 81.8 | (9.8) |
| median (IQR) | 90 | (90-90) | 86.5 | (72-90) |
| Urine protein/creatinine ratio at | | | | |
| screening, mg/mmol | | | | |
| mean (SD) | 191 | (157) | 183 | (82.5) |
| median (IQR) | 164 | (123-180) | 162 | (148-183) |
| Serum albumin at randomisation, g/L | | | | |
| mean (SD) | 33.6 | (5.4) | 37.5 | (6.8) |
| median (IQR) | 33.5 | (32-37) | 37 | (33-43.5) |

^{‡} Mean (SD)
^{§} Ethnicity was reported by the patient
^{††}Screening refers to the first screening visit before rituximab
^{¶}Numerical BILAG-2004 (British Isles lupus assessment group - 2004), where BILAG-2004
A score = 12, B score = 8, C score = 1, and D/E score = 0.
IgG = Immunoglobulin G, IQR = Interquartile range, SD = standard deviation, SLEDAI-2K = systemic lupus erythematosus disease activity index 2000

TABLE 5
Baseline demographics and disease characteristics of the validation cohort

| | | | Active | | Active | |
|---|---|---|---|---|---|---|
| | Healthy | | renal | | mucocutaneous | |
| | donor | | disease | | disease | |
| | (n = 11) | | (n = 6) | | (n = 7) | |
| Age, years ^{‡} | 36.3 | (7.2) | 34.5 | (6.5) | 39.1 | (11.2) |
| Female - no. of participants (%) | 6 | (54%) | 6 | (86%) | 8 | (89%) |
| Ethnicity - no. of participants (%) ^{§} | | | | | | |
| White | 7 | (64%) | 2 | (29%) | 3 | (33%) |
| Black | 0 | (0%) | 4 | (57%) | 3 | (33%) |
| South Asian | 3 | (27%) | 0 | (0%) | 2 | (22%) |
| Chinese | 1 | (9%) | 1 | (14%) | 1 | (11%) |

| Disease duration, years | | | | | |
|---|---|---|---|---|---|
| mean (SD) | — | 7.3 | (5.5) | 6.1 | (6.3) |
| median (IQR) | — | 6.3 | (2.4-11.2) | 6 | (2.3-10.3) |
| Concomitant immunosuppressant or | | | | | |
| prednisolone, no. of patients (%) | | | | | |
| Mycophenolate | — | 3 | (43%) | 2 | (22%) |
| Azathioprine | — | 3 | (43%) | 4 | (44%) |
| Methotrexate | — | 0 | (0%) | 3 | (33%) |
| Prednisolone | — | 7 | (100%) | 6 | (67%) |
| Any concomitant immunosuppressant | — | 6 | (86%) | 9 | (100%) |
| Receiving concomitant immunosuppressant | — | 7 | (100%) | 9 | (100%) |
| or prednisolone | | | | | |
| Concomitant hydroxychloroquine, no. of | — | 5 | (71%) | 8 | (89%) |
| patients (%) | | | | | |
| Average daily prednisolone dose at | | | | | |
| screening, mg/day | | | | | |
| mean (SD) | — | 13.2 | (8.8) | 10.1 | (7.2) |
| median (IQR) | — | 10.2 | (4-14) | 9 | (5-13) |
| Positive anti-dsDNA antibody IgG | — | 7 | (100%) | 9 | (100%) |
| antibody, no. of patients (%) | | | | | |
| Low complement C3 at screening, no. of | — | 7 | (100%) | 8 | (89%) |

patients (%)

^{‡} Mean (SD)
^{§} Ethnicity was reported by the patient
IgG = Immunoglobulin G, IQR = Interquartile range, SD = standard deviation

1. IgA2 antibodies for use as a biomarker.

2. IgA2 antibodies for use as a biomarker in therapy.

3. IgA2 antibodies for use as a biomarker in the treatment or diagnosis of an autoimmune disease.

4. The IgA2 antibodies of any of claims 1-3, wherein the antibodies are IgA2 anti-dsDNA antibodies.

5. A method of identifying a subject with an autoimmune disease who is likely to benefit from treatment with an agent which targets B-cell Activating Factor (BAFF), comprising:
i. providing a biological sample obtained from the subject;
ii. determining the level of IgA2 autoantibodies in the sample;
iii. using the results from step (ii) to determine if the subject is likely to benefit from treatment with an agent which targets BAFF.

6. The method of claim 5, wherein the subject is likely to benefit from treatment with an agent which targets B-cell Activating Factor (BAFF) alone, or
optionally wherein the subject is likely to benefit from treatment with an agent which targets B-cell Activating Factor (BAFF) and an agent which targets CD20.

7. An agent which targets B-cell Activating Factor (BAFF) for use in the treatment of an autoimmune disease in a subject who is identified as likely to benefit from treatment, optionally wherein the subject is identified as likely to benefit from treatment using the method of claim 5 or claim 6.

8. A method of treating a subject with an autoimmune disease, comprising:
i. providing a biological sample obtained from the subject;
ii. determining the level of IgA2 anti-dsDNA antibodies in the sample;
iii. using the results from step (ii) to determine if the subject is likely to benefit from treatment with an agent which targets BAFF; and
iv. administering an agent which targets BAFF to the subject.

9. The method of claim 8, wherein the method further comprises administering an agent which targets CD20 to the subject.

10. The method of any of claim 5, 6 or 8, wherein in step (iii) a subject is determined as likely to benefit from treatment with an agent which targets BAFF, or from treatment with an agent which targets BAFF and an agent which targets CD20, when the level of IgA2 anti-dsDNA antibodies is about two-fold or more higher than the level of IgA2 anti-dsDNA antibodies in a reference sample.

11. The method of any of claim 5, 6 or 8, wherein in step (iii) a subject is determined as likely to benefit from treatment with an agent which targets BAFF, or from treatment with an agent which targets BAFF and an agent which targets CD20, when an Optical Density (OD) of about 0.19 or more is calculated for the level of IgA2 anti-dsDNA antibodies in a sample when measured by ELISA.

12. The method of any of claim 5, 6 or 8, wherein in step (iii) a subject is determined as likely to benefit from treatment with an agent which targets BAFF, or from treatment with an agent which targets BAFF and an agent which targets CD20, when an Optical Density (OD) of about 0.3-0.8 or more is calculated for the level of IgA2 anti-dsDNA antibodies in a sample when measured by ELISA.

13. The method of claim 6 or any of claims 9-12, wherein the agent which targets CD20 is administered before, after, or at the same time as the agent which targets CD20.

14. The method of any of claim 5, 6 or 8-12, wherein the sample is a blood sample, a serum sample, or a urine sample.

15. A method of treating an autoimmune disease in a subject having an increased level of IgA2 anti-dsDNA antibodies comprising: administering to the subject a therapeutically effective amount of an agent which targets BAFF.

16. A method of treating an autoimmune disease in a subject, wherein the method comprises identifying a subject having an increased level of IgA2 anti-dsDNA antibodies and administering to the identified subject a therapeutically effective amount of an agent which targets BAFF.

17. A method of treating an autoimmune disease in a subject classified as a responder, wherein a responder is characterised by having an increased level of IgA2 anti-dsDNA antibodies, comprising administering to the subject a therapeutically effective amount of an agent which targets BAFF.

18. The method of any of claims 8-18, wherein the subject is administered simultaneously, sequentially, or separately, an agent which targets CD20.

19. The method or use of any of claims 3-18, wherein the autoimmune disease is systemic lupus erythematosus (SLE).

20. The method or use of any of claims 3-19, wherein the agent which targets BAFF is an antigen binding polypeptide.

21. The method of claim 20, wherein the antigen binding polypeptide comprises:
a) a heavy chain variable domain comprising:
i. a CDR1 comprising or consisting of SEQ ID NO: 1;
ii. a CDR2 comprising or consisting of SEQ ID NO: 2; and
iii. a CDR3 comprising or consisting of SEQ ID NO: 3, and
b) a light chain variable domain comprising:
i. a CDR1 comprising or consisting of SEQ ID NO: 4;
ii. a CDR2 comprising or consisting of SEQ ID NO:5; and
iii. a CDR3 comprising or consisting of SEQ ID NO:6,
or a sequence with at least about 90% or more, such as 90%, 95%, 98%, 99% identity to one or more of SEQ ID NOs: 1-6.

22. The method of claim 20 or 21, wherein the antigen binding polypeptide is the antibody belimumab.

23. The method of any of claim 6 or 9-22, wherein the agent which targets CD20 is an antigen binding polypeptide.

24. The method of claim 23, wherein the agent which targets CD20 is an anti-CD20 antibody selected from the group consisting of rituximab, ocrelizumab, ofatumumab, ublituximab, veltuzumab, obinutuzumab, ocaratuzumab, PRO131921, tositumomab, and ibritumomab.

25. The method of claim 24, wherein the anti-CD20 antibody is rituximab.
